(19) United States
(12) Patent Application Publication
(10) Pub. No.: US 2016/0047003 A1
(43) Pub. Date: Feb. 18, 2016

(54) HIGH THROUGHPUT METHOD OF SCREENING A POPULATION FOR MEMBERS COMPRISING MUTATION(S) IN A TARGET SEQUENCE

(71) Applicant: VINELAND RESEARCH AND INNOVATION CENTRE, Vineland Station (CA)

(72) Inventors: Travis Wilfred Banks, Fonthill (CA); Daryl John Somers, Fenwick (CA)

(73) Assignee: VINELAND RESEARCH AND INNOVATION CENTRE, Vineland Station, ON (CA)

(21) Appl. No.: 14/773,643

(22) PCT Filed: Mar. 6, 2014
(86) PCT No.: PCT/CA2014/050177

§ 371 Date: Sep. 8, 2015

Related U.S. Application Data
(60) Provisional application No. 61/775,095, filed on Mar. 8, 2013.

(51) Int. Cl. C12Q 1/68

(52) U.S. Cl. CPC C12Q 1/6895; C12Q 2600/156; C12Q 2600/13

(57) ABSTRACT

The present invention provides high-throughput methods of screening for members of a population comprising mutation(s) in one or more target sequence(s). The methods may comprise the steps of: pooling genomic DNA isolated from each member of said population; amplifying the one or more target sequence(s) in the pooled genomic DNA; pooling the amplification products of step (b) to create a library of amplification products; sequencing the amplified products by pair-end sequencing to produce paired-end reads for each sequencing reaction or obtaining paired-end sequence reads for the amplified products; merging the paired-end reads into composite read(s); mapping the composite read(s) to reference sequence(s) to identify mutation(s) in the one or more target sequence(s); and identifying member(s) of the population comprising one or more of the identified mutations in the target sequence(s). The invention further provides kits for use with the methods.

### FIELD OF THE INVENTION

The present invention pertains to the field of molecular biology and genetics. In particular, the present invention relates to high-throughput methods of screening for members of a population comprising mutation(s) in one or more target sequence(s). The invention further provides kits for use with the methods.

### BACKGROUND OF THE INVENTION

The global agriculture industry faces many challenges and pressures that are particularly evident in the production of sessile organisms: biotic and abiotic stresses threatening yield and quality; increasing labour, water and energy costs; and further constraints are imposed by consumer preference. As such, there is great demand to produce crops that are stress tolerant, require little or no input (i.e. reduced use of water, fertilizer, and/or pesticides), and are appealing to consumers at the same time. The possibilities for trait development using traditional breeding are becoming increasingly limited due to a lack of genetic diversity in cultivated plant varieties. Introgression of valuable traits from wild accessions is possible, but this approach might not be feasible if the trait of interest is closely linked to those associated with undesirable traits (Fitzpatrick et al., Plant Cell. 24:395-414).

A transgenic approach can be pursued, but genetically-modified organisms, particularly those yielding edible products, are controversial and present entirely new challenges with respect to food safety regulations and consumer acceptance. Mutagenesis is an effective and efficient method to introduce genetic diversity in crop plants (Wang et al., Plant Biotechnology Journal 10:761-772). The application of random mutagenesis in a Targeted Induced Local Lesions In Genomes (TILLING) approach allows for rational trait design and development, as one can identify plants harbouring lesions in genes known or suspected to be involved in certain biological processes that control a trait of interest. These plants can then be tested to determine if they exhibit the desired phenotype. Therefore, the TILLING technique ultimately promotes translational research in agriculture, by facilitating the transformation of basic research findings into novel traits for the industry. Conveniently, chemical mutagenesis can be applied to essentially any plant system, regardless of genomic resources available for the organism. This approach is particularly appealing to the horticulture industry because of numerous and diverse species cultivated, and the limited genomic resources available for most of these systems.

High-Resolution DNA Melting (HRM) has been used in TILLING approaches for mutation detection in EMS-treated populations (Gady et al., Plant Methods 5:13), however this approach is labour intensive and expensive.

Next generation DNA sequencing (NGS) is an appealing tool to identify mutations in populations of individuals. The rapidly falling price, ever increasing throughput and complete DNA characterization of the sequencing targets has drawn researchers to investigate NGS as a TILLING tool (Rigola et al, PLoS One 4:e4761; Tsai et al., Plant Physiology 156:1257-1268). However, due to the intrinsic error-rate of NGS technologies it is difficult to discern mutation from sequencing mistakes in pools of thousands of individuals. Illumina sequencing technology produces a base-calling error almost twice every 1000 bases sequenced (Minoche et al., Genome Biology 12:R112). In an effort to differentiate errors from mutation, researchers have created multi-dimensional pooling strategies combined with DNA barcoding to sequence members of a population in multiple, independent reactions. Individuals harbouring a mutation are then determined by pool deconvolution using the barcodes (Rigola et al., PLoS One 4:e4761; Missirian et al., BMC Bioinformatics 12:287; WO2007037678 to KeyGene N.V.).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide high-throughput methods of screening a population for members comprising mutation(s) in one or more target sequence(s). In accordance with an aspect of the present invention, there is provided a method for isolation of a member of a population which has one or more mutation(s) in one or more target sequence(s), comprising the steps of: (a) pooling genomic DNA isolated from each member of said population; (b) amplifying the one or more target sequence(s) in the pooled genomic DNA; (c) pooling the amplification products of step (b) to create a library of amplification products; (d) sequencing the amplified products by paired-end sequencing to produce paired-end reads for each sequencing reaction or obtaining paired-end sequence reads for the amplified products; (e) merging the paired-end reads into composite read(s); (f) mapping the composite read(s) to reference sequence(s) to identify mutation(s) in the target sequence(s); and (g) identifying member(s) of the population comprising one or more of the identified mutations in the target sequence(s). In certain embodiments, the member(s) of the population comprising one or more of the identified mutations in the target sequence are identified by high-resolution DNA melting (HRM).

In accordance with another aspect of the invention, there is provided a method for identifying one or more mutation(s) in one or more target sequence(s) in a population, comprising the steps of: (a) pooling genomic DNA isolated from each member of said population; (b) amplifying the one or more target sequence(s) in the pooled genomic DNA; (c) pooling the amplification products of step (b) to create a library of amplification products; (d) sequencing the amplified products by pair-end sequencing to produce paired-end reads for each sequencing reaction or obtaining paired-end sequence reads for the amplified products; (e) merging the paired-end reads into composite read(s); and (f) mapping the composite read(s) to reference sequence(s) to identify mutation(s) in the one or more target sequence(s).

### BRIEF DESCRIPTION OF THE FIGURES

FIG. 1 provides a flow chart illustrating the steps in one embodiment of the method.

FIG. 2 illustrates the steps to create stoichiometrically balanced amplicon pools for sequencing in one embodiment of the method. The steps for this embodiment of the method are as follows: Step 1: For each 96-well plate in the mutant population pool equimolar amounts of DNA from each well are added into a single tube to form plate pools. A worker skilled in the art would appreciate that the amount of DNA depends on how many amplicons need to be created. Step 2: For each amplicon: 5 independent PCR reactions using DNA from the plate pool as template are performed. The 5 finished PCR reactions are pooled into a single tube to form the amplicon pools. This is completed for each plate pool. A small amount of each amplicon pool is run on a gel to determine whether the PCR was successful. If the PCR reaction was not successful, the PCR reactions for that plate pool is redone. The concentration of each amplicon pool is then determined. Step 3: For each amplicon: Equimolar amounts of amplicon pools are pooled in groups of four to represent a 384-well plate in a single tube to form 384-well amplicon pools. Each 384-well amplicon pool sample is run through a PCR cleanup column to both clean and concentrate the sample. The concentration of each 384-well amplicon pool is determined. After the preceding two steps have been done for each amplicon the library pool is produced. Step 4: Produce the library pool—this step allocates the 384-well amplicon pools among library pools. A library pool will contain one or more 384-well amplicon pools for each amplicon to be screened. Amplicons within a library pool are aliquotted in equimolar amounts.

FIG. 3 illustrates the steps for processing the data to produce high quality composite sequences in one embodiment of the method. The steps are as follows: From the library pools PE Illumina reads with an average insert size close to the read length of the Instrument are created (100 bp). This is to maximize overlap between forward and reverse reads. SHERA is used to create composite reads with enhanced quality scores. This dramatically reduces errors due to miscalling during the sequencing reaction. RepeatMasker is used to mask adapter and primer fusions that cutadapt cannot process. Cutadapt is used to remove primer, adapter and Illumina library barcodes. Two step processes of masking all base calls with a quality score not supported by PE reads (Q>=60). Following masking 5′ and 3′ strings of the masking character ‘N’ are removed.

FIG. 4 illustrates the steps for variant (or mutation) identification in one embodiment of the method. The steps are as follow: De Novo Assembly: If a reference sequence doesn't exist for the gene under investigation perform a de novo assembly of the DVS data to create one. Read Mapping: Align the HQ composite reads to a reference sequence. Bowtie2 with high stringency settings may be used. Positional Tally: Using SAMTools and Perl the occurrence of the 4 bases at each reference position are counted. Statistical Weighting: The distribution of non-reference base call counts forms a normal distribution. Each alternative base for a position is assigned a p-value based on the distribution. Mutant Identification: Mutations are selected based on predicted effect of the mutation and p-value. HRM is used to genotype our mutant population for plants with mutations of interest. The breadth of the search is limited by identifying the 384-library containing each mutation.

FIG. 5 provides a cost comparison of mutation screening methods and services. This figure illustrates the costs associated with screening five, 2 kb DNA fragments in a population of 2000 M1 families (12,000) individuals. DVS is the method of an embodiment of the invention).

FIG. 6 provides the sequence of three target regions interrogated by one embodiment of the method.

### DETAILED DESCRIPTION OF THE INVENTION

Targeting Induced Local Lesions in Genomes (TILLING) is a method for identification of mutations in a specific gene and has been applied to a broad range of organisms and cells, including but not limited to plants, yeast, insects such as fruit flies, birds and mammals such as mice. Typically, the method combines the creation of a structured population of individuals that have had their DNA randomly mutated by chemical means (such as ethyl methanesulfonate (EMS)) or physical means (such as ionizing radiation (fast neutron bombardment)) with screening of the mutagenized population for individuals harbouring one or more mutations in the target gene (McCallum et al., Nat. Biotechnol 18:455-457; McCalmm at al., Plant Physiology 123:439-442; Till et al. Genome Research 13:524-530; Li at al., The Plant Journal 27:235-42).

Every individual (such as an individual plant) in the mutagenized population carries several hundred (or thousand) mutations, some of which affect normal development, growth, morphology or otherwise confer a phenotype due to loss-of-function (knock-out, knock-down) of one or multiple genes or their regulatory sequences. A TILLING population generally contains a sufficient number of individuals to cover all genes with multiple independent mutations (5-20 per gene). A mutagenized plant population used in TILLING therefore usually consists of 2000-5,000 individuals.

The mutagenized population is screened for individuals harbouring mutations in a target sequence. The target sequence may be selected following analysis of the scientific literature and/or experimentation for sequences or genes of interest. The individual members of the population harbouring mutations in the target sequence are then grown and subjected to phenotypic evaluation. TILLING methods may also be used in non-mutagenized populations to screen for naturally occurring mutations in a given population.

A number of approaches may be used to screen mutations in TILLING populations. These methods include but are not limited to methods based on mismatch cleavage by enzymes such as CEL I, mung bean nuclease, S1 nuclease; methods based on heteroduplex detection using DNA High Resolution Melting (HRM); methods using traditional Sanger sequencing, and methods utilizing next-generations sequencing (NGS).

Despite their high throughput the most popular NGS technologies (Illumina and Roche 454) generate an error more than 0.1% of the time. In order to address this error rate, an approach using multidimensional pooling which structures the population's DNA such that DNA from each individual is present in at least two dimensional pools (row, column) that are independently processed was previously developed. This method involves uniquely tagging fragments for each dimensional pool. A sequence variant has to be present in a least 2 pools to proceed. The pool tags are then used to identify the sample which contained the variant DNA.

Described herewith is a new method for isolation of a member of a population which has mutation(s) in one or more target sequence(s) that uses composite sequences from overlapping paired-end reads to reduce the effective error rate caused by NGS for identifying sequence variants in pools of genetically distinct individuals. This method allows for thousands of individuals to be interrogated simultaneously without dimensional pooling and tagging. After identifying variants of interest that exist in the population, DNA High Resolution Melting may be used to genotype the population to identify individual population members carrying the mutation(s).

The method comprises (a) pooling genomic DNA isolated from each member of said population; (b) amplifying region(s) within one or more target sequence(s); (c) pooling the amplification products of step (b) to create a library of amplification products; (d) sequencing the amplified products by pair-end sequencing to produce paired-end reads for each sequencing reaction or obtaining paired-end sequence reads for the amplified products; (e) merging the pair-end reads into composite read(s); (f) mapping the composite read(s) to reference sequence(s) to identify mutations in the one or more target sequence(s); and (g) identifying member(s) of the population comprising one or more of the identified mutations in the one or more target sequence(s).

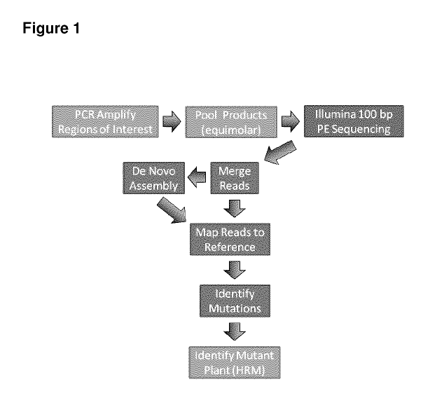

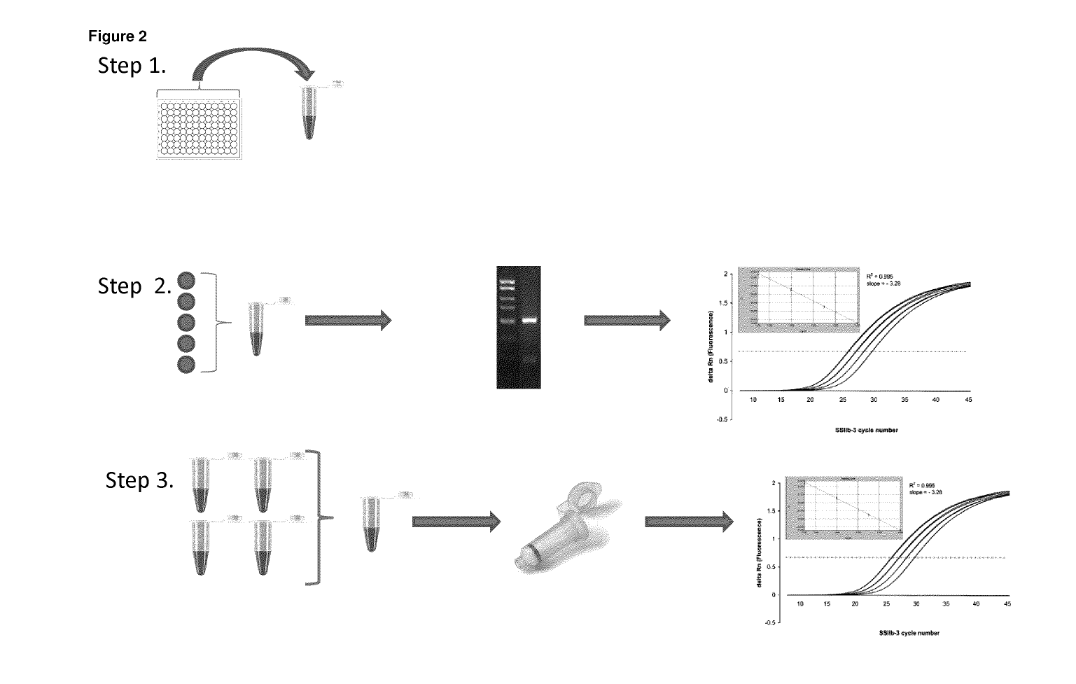

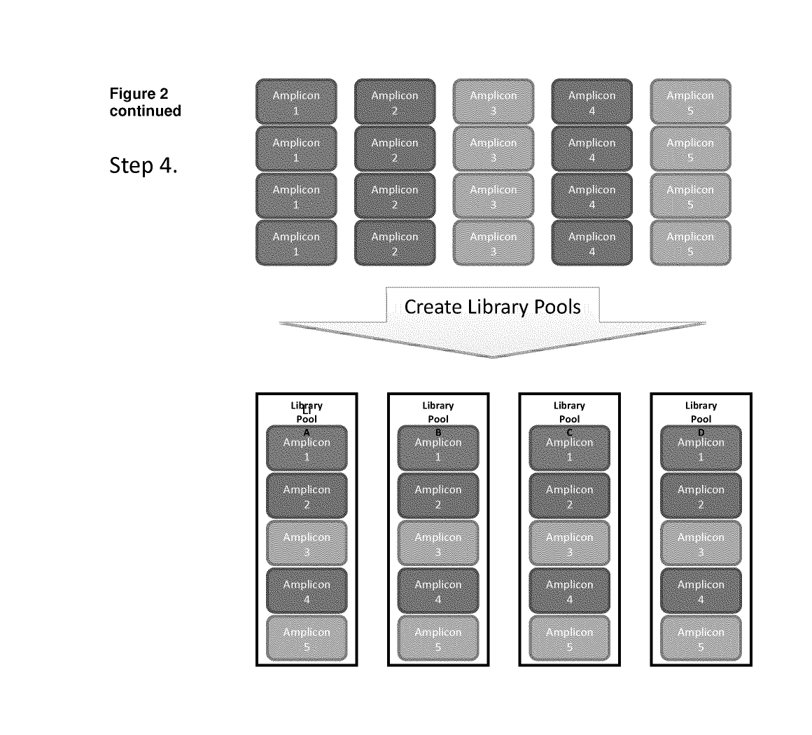

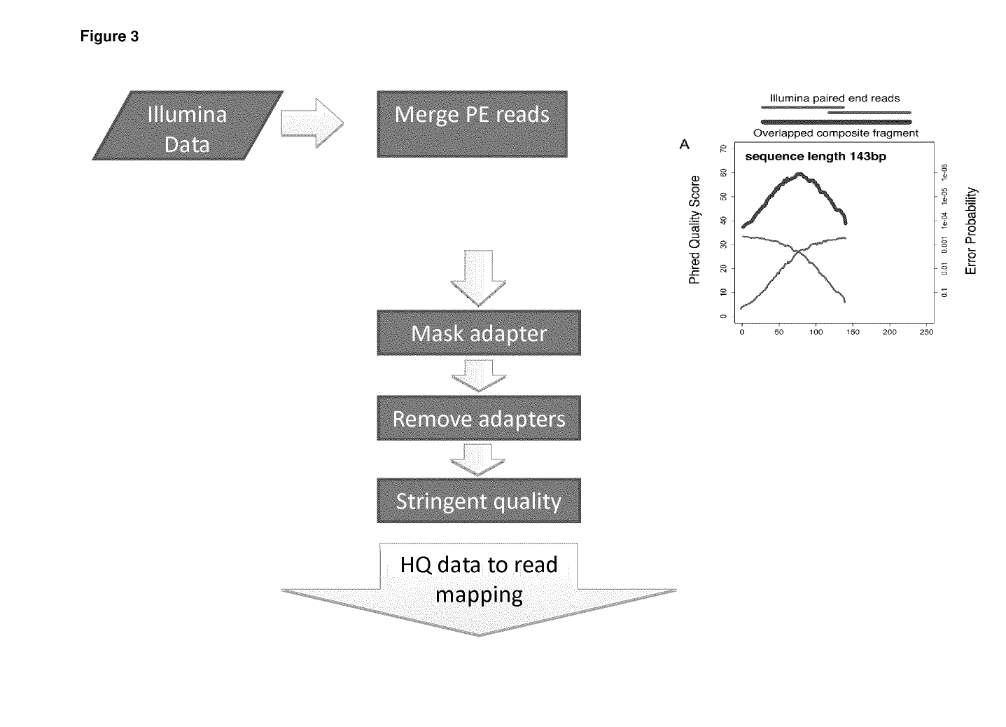

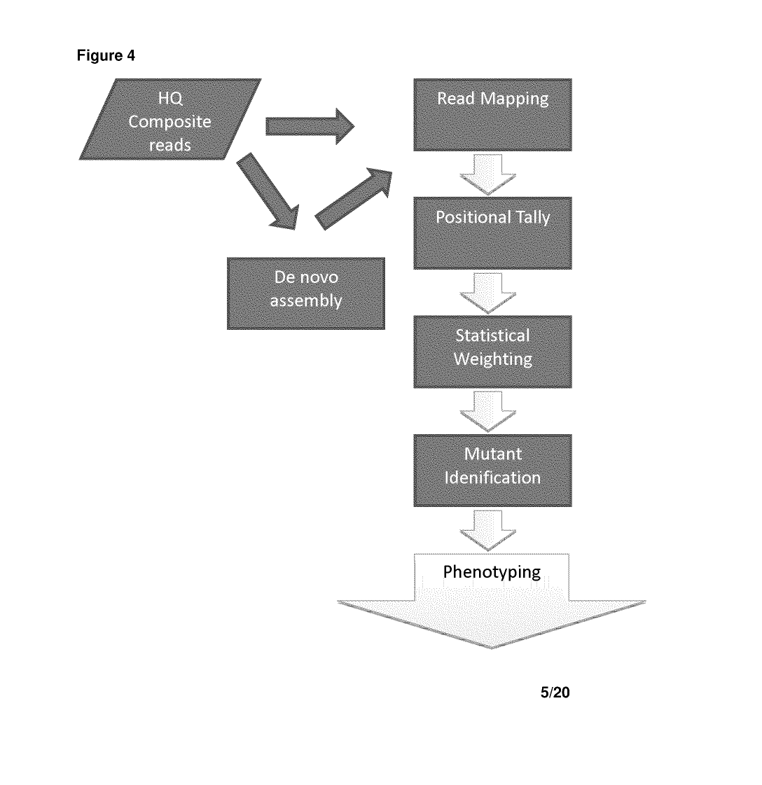

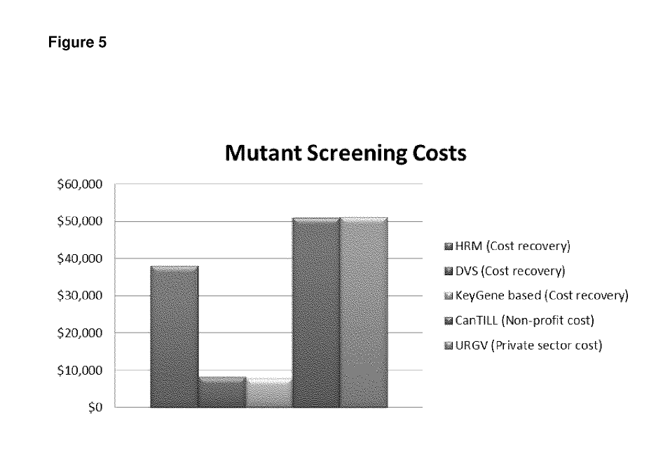

In one embodiment, the method comprises the steps as set forth in FIG. 1. In another embodiment, the method comprises the steps as set forth in FIGS. 2 to 4.

### Population

The population from which the genomic DNA is isolated may be a non-mutagenized population, mutagenized or transgenic population of organisms and the progeny thereof (including but not limited to plants or cells). The population may be plants, cells or animals such as Drosphila or mice. The plants may be, for example, a grain crop, oilseed crop, fruit crop, vegetable crop, a biofuel crop, an ornamental plant, a flowering plant, an annual plant or a perennial plant. Examples of plants include but are not limited to petunia, tomato (Solanum lycopersicum), pepper (Capsicum annuum), lettuce, potato, onion, carrot, broccoli, celery, pea, spinach, impatiens, cucumber, rose, sweet potato, apple and other fruit trees (such as pear, peach, nectarine, plum), eggplant, okra, corn, soybean, canola, wheat, oat, rice, sorghum, cotton and barley. In certain embodiments, the population is a variety of annuals. In specific embodiments, the population is a population of petunias.

A worker skilled in the art would readily appreciate that mutations may occur spontaneously in a population or the population may be mutagenesized by chemical means or physical means. For example, a worker skilled in the art would readily appreciate that ethylmethane sulfonate (EMS) may be used as a mutagen or ionizing radiation, such as x-ray, y-ray and fast-neutron radiation may be used as a mutagen. A worker skilled in the art would readily appreciate that the population may be subjected to targeted nucleotide exchange or region targeted mutagenesis. A worker skilled in the art would further appreciate that transposable elements can act as mutagens.

In certain embodiments of the invention, the population is a population of plants mutagenesized with EMS.

In certain other embodiments, the population is a population of Petunia x hybrid mutagenesized with EMS.

In other embodiments, the population may have been genetically engineered. A worker skilled in the art would readily appreciate methodologies for genetically engineering a population.

### Identification of a Target Sequence

The candidate target sequence(s) is identified through analysis of the scientific literature and/or experimentation. Typically, a target sequence is a region of a gene that a mutation would have an effect. For example, a worker skilled in the art would readily appreciate that mutations in non-coding sequences, such as introns, may have little or no effect. Such a worker would further appreciate that mutations in conserved coding regions of genes have an increased likelihood of having an effect. CODDLE (Codons to Optimize Discovery of Deleterious Lesions; www.proweb.org/coddle/) is a web based program which may be used identify regions where point mutations are most likely to have effects. Typically, a target sequence is greater than 1000 bases in length to facilitate fragmentation during sequencing library preparation. In cases where the target sequence is greater than the longest PCR amplicon possible with the chosen DNA polymerase, multiple PCR amplicons are created. In cases where multiple PCR amplicons are necessary, the PCR amplicons will overlap no less than 200 bp.

In embodiments in which multiple target sequences are examined, each of the target sequences may be in the same or different genes. For example, in embodiments where two target sequences are examined, both target sequences may be in the same gene or the first target sequence may be in a first gene and the second target sequence may be in a second gene. Accordingly, in certain embodiments, one or more genes are screened for mutations. In certain embodiments, two or more genes are screened for mutations. In certain embodiments, three or more genes are screened for mutations.

### Isolation of Genomic DNA

Methods of isolation of genomic DNA are known in the art. A worker skilled in the art would readily appreciate that the quality of the genomic DNA impacts TILLING and, as such, protocols which produce high quality genomic DNA with minimal contamination are preferable. In addition, a worker skilled in the art would readily appreciate that kits for isolation of genomic DNA are commercially available (for example Purelink™ Genomic Kit from Invitrogen or Wizard® Genomic DNA Purification Kit from Promega).

### Pooling of Genomic DNA

Typically, with TILLING methodologies, equimolar amounts of genomic DNA from a number of the members of the population are pooled to produce a sample pool. Often this pooling is of multiple siblings from the same parents. In order to facilitate high-throughput TILLING procedures have been adapted to multi-well plates, such as 96 well plates (Till et al. Genome Research 13:524-530).

Equimolar amounts of genomic DNA from each sample are pooled. In one embodiment, equimolar amounts of genomic DNA from each well of a 96 well plate are pooled to create a pool plate. In another embodiment, equimolar amounts of genomic DNA from each well of a 384 well plate are pooled to create a pool plate. A worker skilled in the art would readily appreciate that the amount of DNA from each sample will be dependent upon how many amplicons are needed. In certain embodiments, in order to reduce the impact of early stage DNA polymerase errors, at least 30 diploid genome copies of each individual in a well are used in a single PCR reaction.

In certain embodiments, greater than 50 genome copies from each individual in a well are pooled. A worker skilled in the art could readily determine the amount of DNA. For example, for petunia, at least 30 genome copies of each individual plant is ˜50 ng for petunia assuming 6×96 individual plants in each PCR reaction.

Amplifying Regions within the Target Sequence

The pooled genomic DNA is used as a template for polymerase chain reactions (PCR) which produce amplicons for one or more target sequence(s). Each PCR reaction preferentially amplifies a single region in the target sequence. As discussed, in detail below, amplicons from different regions of the target sequence may then be combined to produce a library pool.

In order to reduce the number of DNA polymerase errors propagated through the PCR, multiple PCR reactions using DNA from the plate pool may be performed and then pooled together to produce an amplicon pool. Optionally, the PCR reactions are purified (for example, by column purification) prior to combining. In certain embodiments, 3 to 12 PCR reactions are performed using DNA from the plate pool and then pooled together to produce an amplicon pool. In certain embodiments, 5 PCR reactions are performed using DNA from the plate pool and pooled together to produce an amplicon pool. A worker skilled in the art would readily appreciate that DNA polymerase errors may also be minimize by use of a high-fidelity enzyme such as Kapa Taq (Kapa Biosystems), Platinum Taq (Invitrogen), PFUUltra (Agilent Technologies) or Phusion (New England Biolabs).

A worker skilled in the art would readily appreciate methods for determining if the PCR reaction was successful and the amount of DNA produced. In addition, a worker skilled in the art would readily appreciate methods for concentrating and cleaning a PCR sample.

A worker skilled in the art would readily appreciate that not all commercial DNA polymerases are able to polymerize the same length of amplicon and not all regions of DNA are able to be amplified with the same efficiencies. Primers to amplify regions of interest are chosen to maximize the length of target sequence amplified and produce a robust single band when viewed on an agarose gel. Typically, the size of the amplicon ranges from 1000 by to greater than 6500 by depending on the length of the region one is amplifying and the DNA polymerase used. In cases where the region of interest is larger than what can be produced in a single PCR product, the region of interest is amplified as two or more smaller PCR products that overlap. At least 200 by of overlap is generated between amplicons. This is done to compensate for the low sequencing coverage often found at the 5′ and 3′ extremes of the product being sequenced. A worker skilled in the art would appreciate that the PCR conditions used will be dependent on the DNA polymerase used, the primers selected and the quality of the PCR template DNA.

### Pooling the Amplification Products to Create a Library Pool

Multiple amplicon pools may be combined in equimolar amounts to produce a library of amplicon pools which is used to construct a library for use in paired-end sequencing. For example, equimolar amounts of genomic DNA from four 96-well amplicon pools targeting the same region of the target sequence may be combined to produce a 384-well amplicon pool to one region of the target sequence. Alternatively, a single 384-well plate is used to produce the 384-well amplicon pool. Equimolar amounts of a number of these 384-well amplicon pools targeting different regions of the target sequence or different target sequences may then be combined to produce a library pool. In one embodiment, five 384-well amplicon pools are combined to produce the library pool. The number of 384 well plates depends on the population size but can range from 1 to 15 384 well amplicon pools to produce a library pool.

In certain embodiments, a sufficient number of amplicon pools targeting different regions within the target sequence are combined such that the complete target sequence is represented in the library pool. In other embodiments a sufficient number of amplicon pools targeting different target sequences are combined to produce the library pool.

In certain embodiments, equimolar amounts of four 96-well amplicon pools targeting a single region of the target sequence (or single target sequence) are combined to produce a 384-well amplicon pool. In other embodiments, a single 384-well plate is used to produce the 384-well amplicon pool. Equimolar amounts of multiple 384-well amplicon pools targeting different regions of the target sequence or different target sequences are then combined to produce a library pool. In certain embodiments, five 384-well amplicon pools targeting overlapping regions of the target sequence are combined to form the library pool.

A worker skilled in the art would readily appreciate how to concentrate and clean the 384-well amplicon pool prior to combining multiple pools to form the library pool. Methods of preparing a sample such as the library pool for paired-end sequencing are known in the art and kits are commercially available (for example, from Illumina).

In certain embodiments, the average insert size of the library is set to the read length of the sequencing run so that the overlap between the forward and reverse reads is maximized. In certain embodiments, the average insert size of the library is set to 100 base pairs.

Sequencing the amplified products of the Library Pool and Merging the Paired-End Reads into Composite Reads

The library pools are sequenced in a paired-end sequencing assay. Forward and reverse reads are combined into a single composite read. Base calls with an error likelihood of >1/100,000 are removed or masked. In certain embodiments, the paired-end sequencing is conducted by a third party and the paired-end sequencing data is obtained from the third party.

A worker skilled in the art would readily appreciate that a forward and reverse read-pair are independent sequencing reactions over the same template molecule. Such a worker would further appreciate that when base calls from aligned reads agree in both the forward and reverse directions the confidence that the base is called correctly increases. Rodrigue et al. (PLoS One 4:34761) demonstrated that combining the forward and reverse read-pairs from an Illumina paired-end sequencing run reduces the sequencing error-rate by 2-orders of magnitude. With an error rate of 1/100,000 or better, DNA samples from thousands of individuals can be sequenced at once without losing mutations in a sea of noise.

A worker skilled in the art would readily appreciate that there is software available, such as SHERA ((Rodrigue et al, PLoS One 4:34761) or PEAR (Zhang et al., Bioinformatics; PMID 24142950) which may be used to produce composite reads from the paired-end reads. Alternatives to SHERA and PEAR include COPE (Liu et al, Bioinformatics 28(22): 2870-2874, FLASH (Mago\check{c} and Salzberg, Bioinformatics 27(21): 2957-2963), and PANDASeq (Masella et al., BMC Bioinformatics 13:31).

Identification of mutations in the Target Sequence

The composite read(s) are then mapped to one or more reference sequence(s) to identify mutations in the one or more target sequence(s). The reference sequence(s) may be a sequence known in the art or if the complete target sequence is unknown, the composite reads may be assemble to form a complete target sequence.

A worker skilled in the art would readily appreciate that there is software available to map the composite reads to the reference sequence. For example, the software Bowtie2 (http://sourceforge.net/projects/bowtie-bio/files/bowtie2/2.0.2/) may be used to align the composite read(s) to the reference sequence and SAMTools (Li et al., Bioinformatics 25(16):2078-2079) and Perl (http://www.perl.org) may be used to analyze the aligned sequences for mutations. BWA (Li and Durbin, Bioinformatics 25(14):1754-60), MAQ (Li et al., Genome Research 18:1851-1858), MOSAIK (http://bioinformatics.bc.edu/marthlab/Mosaik), and SOAP2 (Li et al, Bioinformatics 25(15):1966-1967) are all software capable of mapping reads to a reference sequence like Bowtie2 but with different speeds and sensitivities.

### Identification of Member(s) of the Population Comprising One or More of the Identified Mutations in the Target Sequence(s).

In one embodiment, High Resolution Melting (HRM) is then be used to identify member(s) of the population comprising the one or more identified mutations in the one or more target sequence(s). Methods of HRM are known to a worker skilled in the art. See, for example, Erali and Witter (Methods 50(4):250-261).

In particular, HRM may be conducted utilizing primers which flank the identified mutation alone or in combination with a 3′ block nucleotide probe (such as ‘LunaProbe’ (as described by Idaho Technology) and the genomic DNA of the individuals of the population, which may or may not be pooled.

In certain embodiments, once the presence of a mutation in a population has been detected using NGS,the individual DNA sample containing the mutation is identified using HRM (De Koeyer et al, Molecular Breeding 25: 67-90). In some embodiments, PCR primers flanking the mutation of interest are created and used to amplify a product containing the mutation site in each of the DNA samples from the 384 well pools where the mutation of interest was identified. The PCR primers can be designed such that the amplicon size is less than 75 by and no naturally occurring heterozygous DNA positions. In certain embodiments, the single DNA sample containing the mutation is identified through melt curve analysis. For example, a 384 well LightScanner (Idaho Technology) and LCGreen Plus HRM dye may be used in the melt curve analysis. Optionally, the presence of the mutation may be confirmed. In certain embodiments, to confirm the mutation, the seed collected from plants contributing DNA to that sample are planted and grown. Tissues are collected from these plants and their DNA analyzed using Sanger sequencing so that individual plants with the mutation are identified.

Optionally, the presence of the mutation may be confirmed in the individual identified through other SNP detection method.

### Phenotypic Analysis

Phenotypic evaluation of plants may be performed to determine if the mutations of interest have an effect on the performance of the plant under various conditions. Types of phenotypic analysis include, but are not limited to, evaluating drought stress responses, low temperature growth, heat tolerance, pathogen resistance, yield, change in morphology (including but not limited to plant height, size and/or colour of leaf, seed and/or flower), modification in life span and/or disease susceptibility.

### Kits

Kits comprising one or more of reagents necessary for the methods set forth therein. For example, the kits may include any of one or more primers, probes, DNA polymerase and other reagents and instructions for use.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Method Simulation and Proof of Concept

To evaluate the effectiveness of the method a computer simulation was performed using the Illumina read simulator pIRS (Hu et al, Bioinformatics 28:1533-1535). An experiment with 12,000 individual petunias (Petunia x hybrida) and two target gene regions totalling 8,000 bases in length was simulated. For each of the target regions, one individual was ‘mutated’ in silco at a position validated empirically as a true mutation in our petunia EMS population.

A virtual sequencing run was established using an average insert size of 130±40 by and 100 million paired-end reads. Using the read mapping software Bowtie2 (Langmead et al, Nature Methods 9:357-359), the reads were aligned to target sequences and SAMtools (Li et al, Bioinformatics 25:2078-2079) was used to generate base counts at all positions along the alignments. Using manual inspection, it was quite evident that SNPs were present at positions of the introduced mutations. A number of SNP-calling software programs [SAMtools (Li et al, Bioinformatics 25:2078-2079); SOAPSNP (http://soap.genomics.org.cn); MAQ (Li et al, Genome Research 18:1851-1858); CLC Genomics Workbench (http://cicbio.com)] were tried, but none of these could detect a SNP with 50× coverage at positions with read depths greater than 500,000×. This represents one mutant individual in a population size of 10,000 in our simulation.

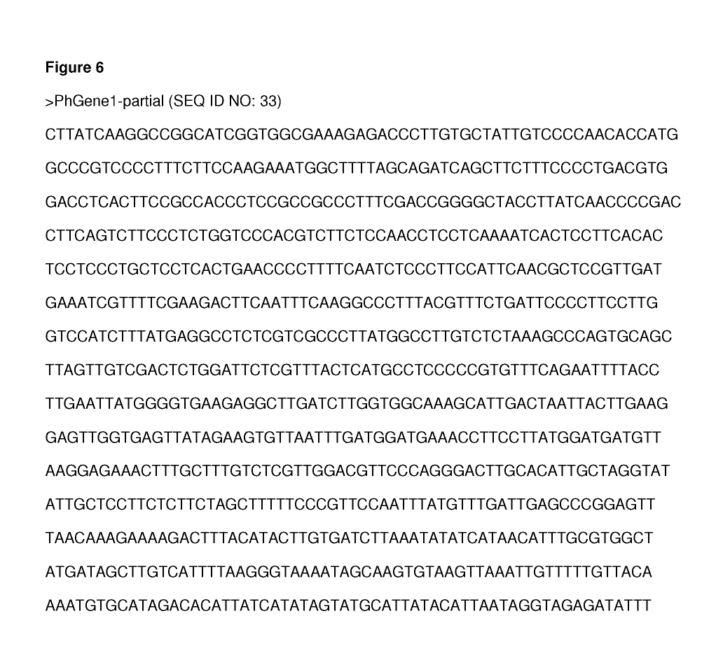

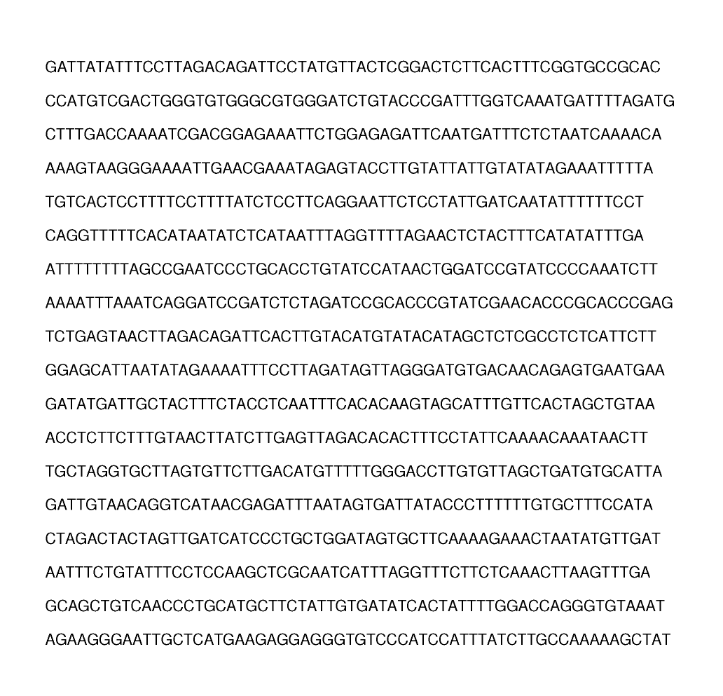

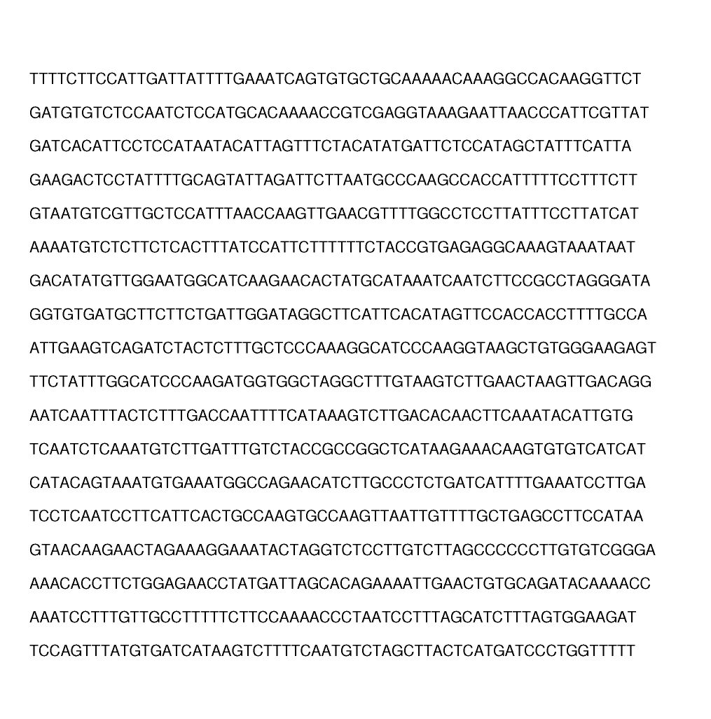

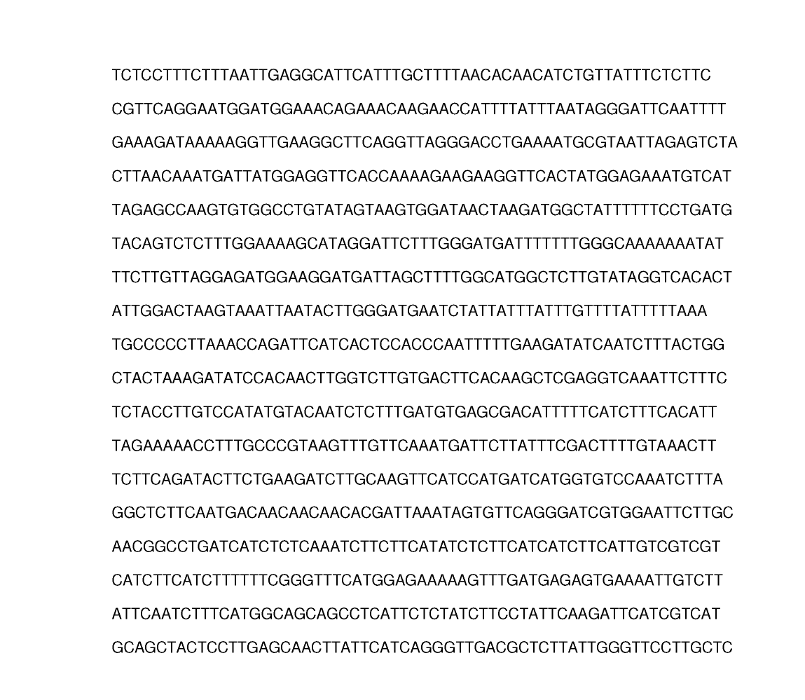

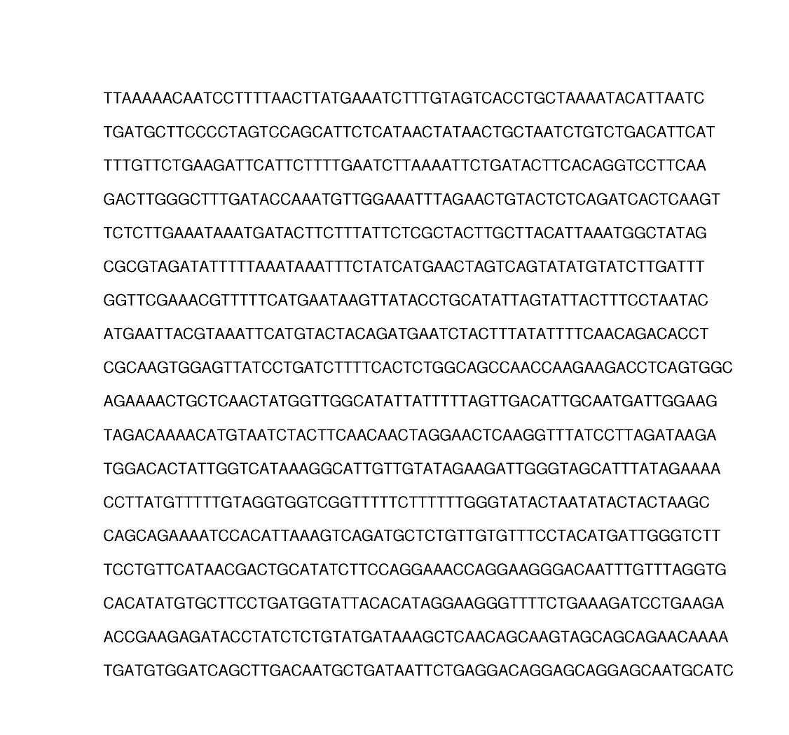

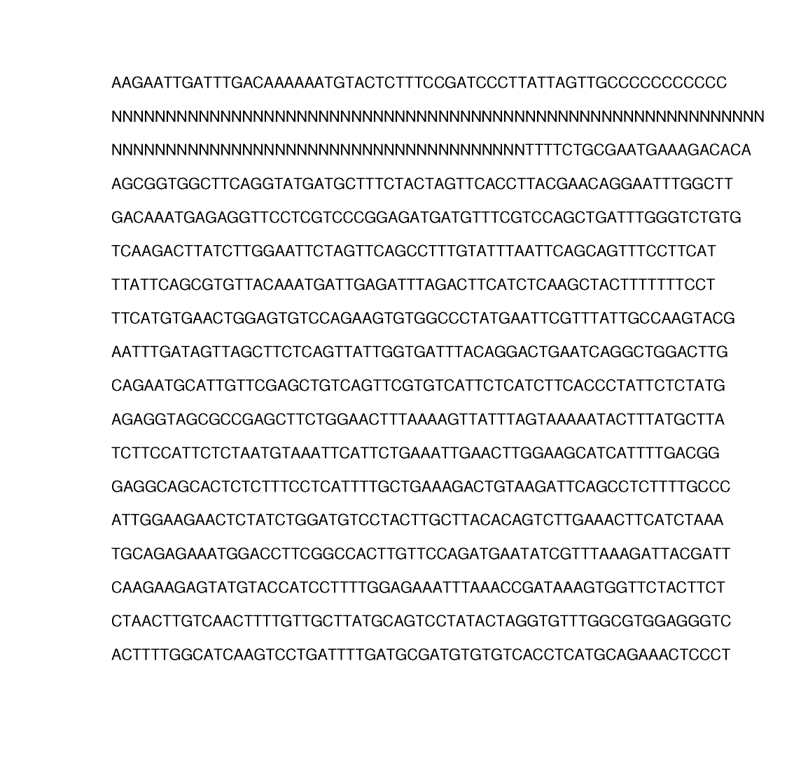

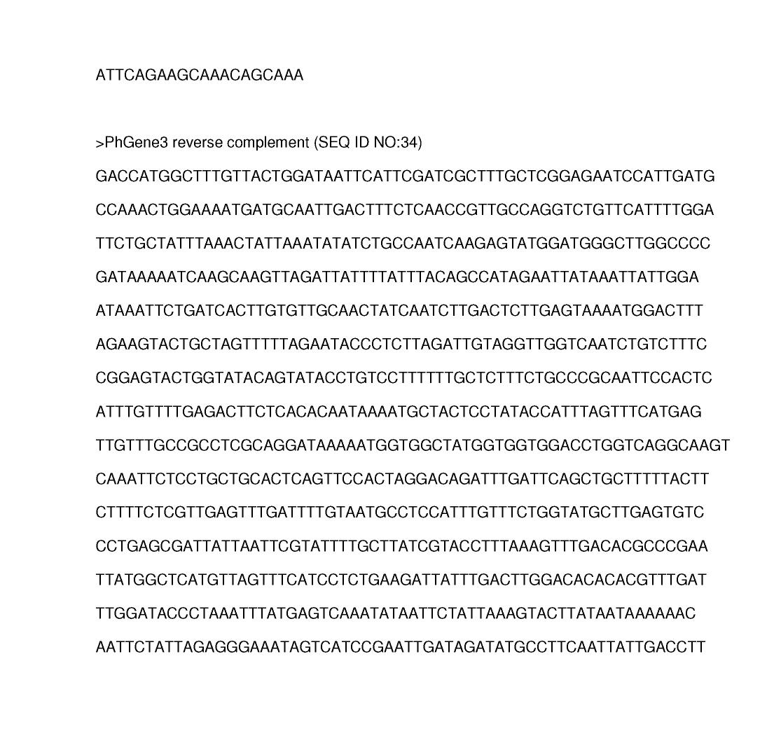

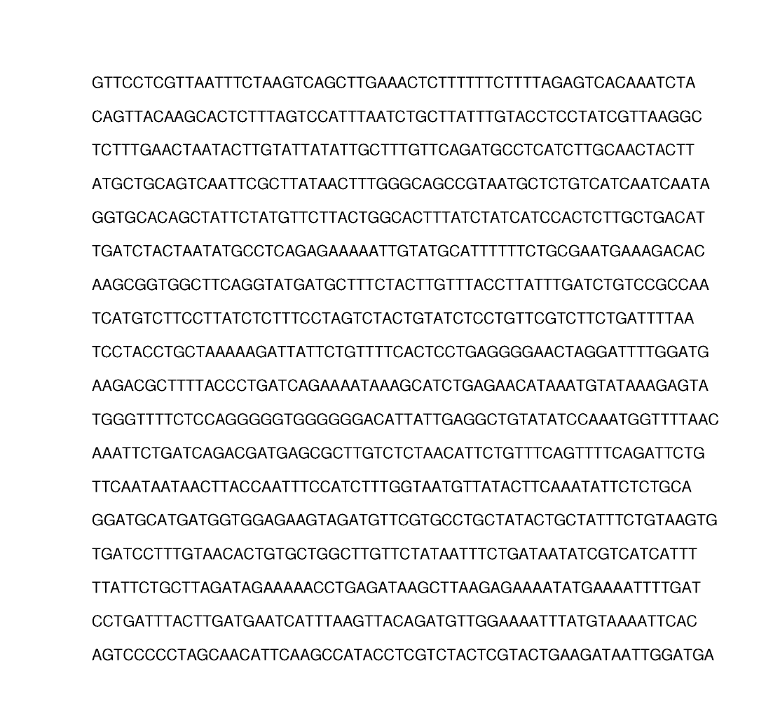

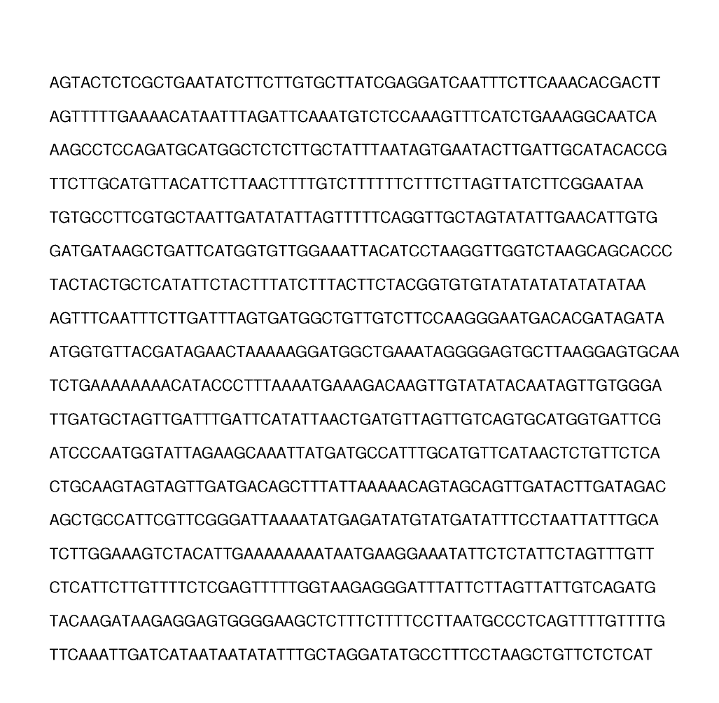

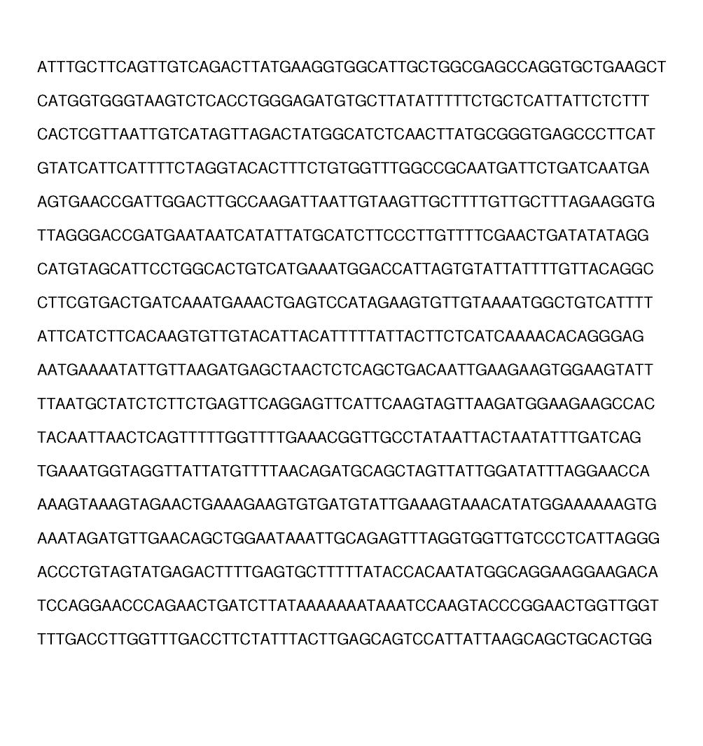

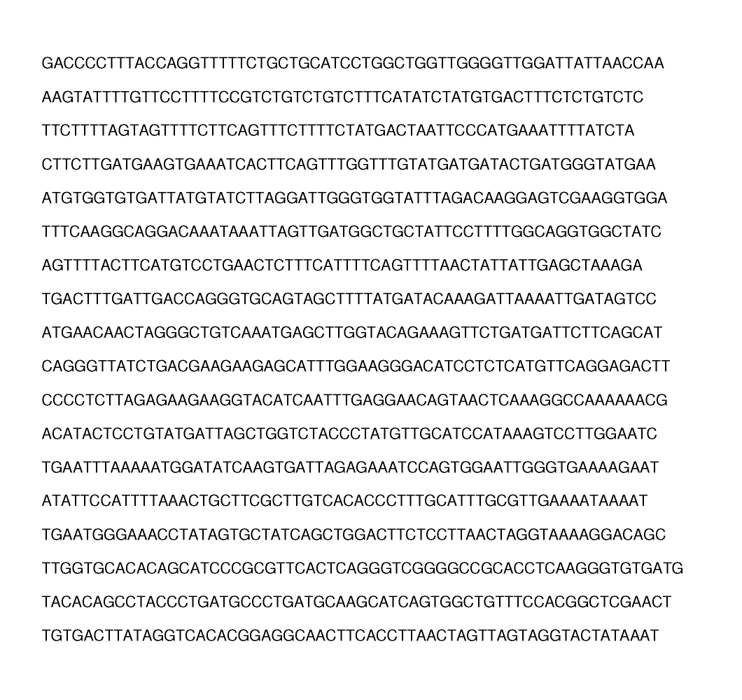

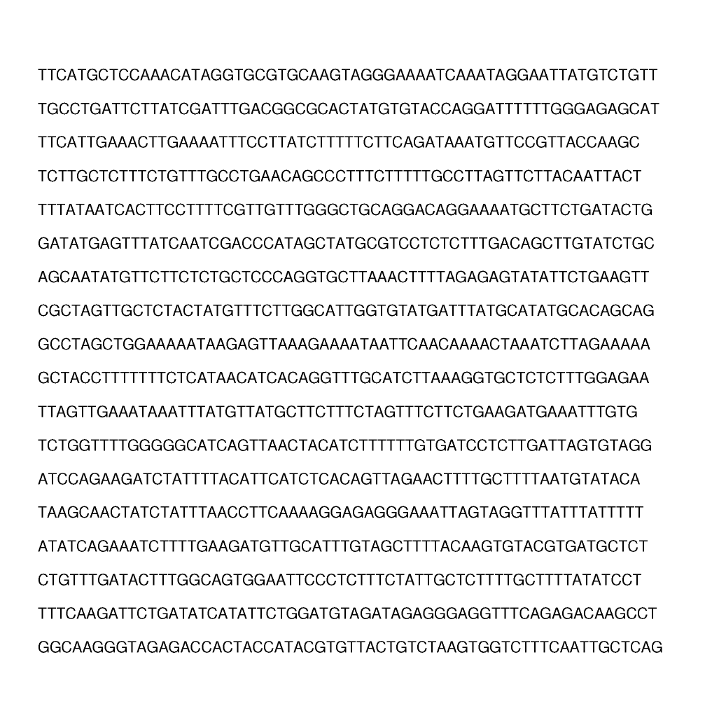

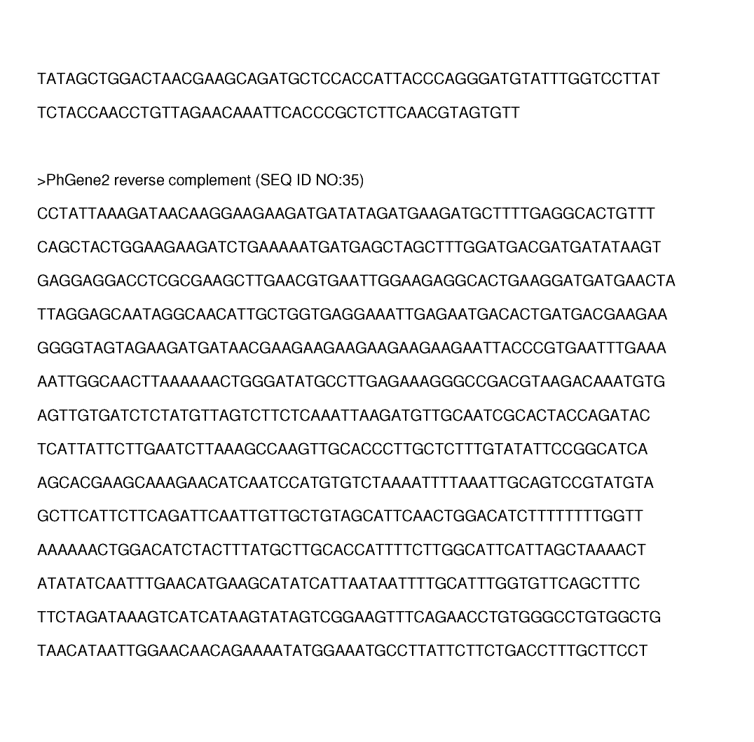

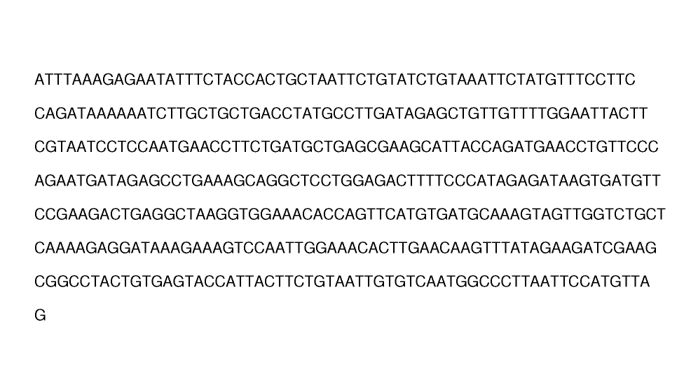

Following the simulation, a proof-of-concept experiment was performed where 3 gene regions totalling ˜14,000 by (FIG. 6) were interrogated for mutations using our method. The method was carried out as set forth in FIGS. 2 to 4, and ˜200 million 100 by paired-end reads were generated on a single HiSeq 2000 lane. In these empirical data, much more noise than the simulation was encountered arising likely from polymerase errors from the PCR amplification stage of the process. Despite this second source of noise, all 12 of the mutations previously identified in this population through HRM screening (positive controls) were identified at p-values ˜1×10⁻² using this method.

### Proof of Concept Target Identification

Three gene targets (see FIG. 6) were identified based on mutant phenotypes observed in Arabidopsis thaliana; PhGene2PhGene1AtGene1, AtGene2, and AtGene3. Reciprocal TBLASTN/BLASTP searches using the protein sequence of the A. thaliana genes against an in-house transcriptome database of Petunia hybrida identified putative P. hybrida orthologs of the A. thaliana targets. Evaluation of the genome sequences of tomato and potato, two relatives of petunia, found each of the three genes to be single copy. We assumed that the P. hybrida sequences were single copy genes as well.

### DNA Isolation

The DNA isolation protocol used in our proof of concept was modified from Kim et al, Nucleic Acids Research 25:1085-1087
1. Tissue was harvested, frozen and lyophilized. The tissue (2×2.5 cm sections) was then placed in 1.2 ml collection tubes with ˜200 ul glass beads (2 mm) and shaken on Qiagen tissue grinder.
2. Extraction Buffer (250 mM NaCl, 25 mM EDTA, 0.5% SDS, 200 mM Tris-HCl pH8.0) was prepared.
   For 1 litre:
      200 ml 1.0M Tris-HCl pH 8.0
      50 ml 0.5M EDTA pH 8.0
      50 ml 10% SDS
      50 ml 5M NaCl
      650 ml ddH₂O
3. The extraction buffer was preheated to 65° C. and the plates containing the tissue was allowed to warm up to room temperature if they have been stored at −20° C.
4. 500u1 of extraction buffer was added to each tube, the plates were sealed with caps and shaken thoroughly. The plate was incubated at 65° C. for 30 minutes and the tubes were shaken every 5 minutes.
5. The plates were placed in the fridge (or freezer) to cool them down to room temperature (about 15 minutes) before 250 μl 6M ammonium acetate (stored at 4° C.)+18% PVP (PVP-10) (for working concentration of 6% per sample after diluted with extraction buffer) was added. The 6M ammonium acetate (stored at 4° C.)+18% PVP (PVP-10) was prepared. The plates were shaken vigorously to mix in the ammonium acetate and then left to stand for 15 minutes in the fridge.
6. The plate was centrifuged for 15 minutes at 5000 rpm to collect the precipitated proteins and plant tissue.
7. 600 μl of the supernatant was recovered into new collection microtubes containing 360 ul of iso-propanol in each well. The microtubes were mixed thoroughly and the DNA was allowed to precipitate for 5 minutes.
8. The samples were centrifuged for 15 minutes at 5000 rpm in order to pellet the DNA and then the supernatant was tipped off. The remaining fluid was allowed to drain off the DNA pellet by inverting the tubes onto a piece of paper towel.
9. The pellet was washed in 500 μl of 70% ethanol.
10. The plate was centrifuged for 15 minutes at 5000 rpm and the supernatant was discarded.
11. The pellets were completely dried in 40-60° C. oven for 30-60 minutes.
12. The pellet was resuspended in 300 μl of 0.1×TE. The DNA was left to dissolve overnight at 4° C. in the fridge.
13. The plate was centrifuged for 20 minutes at 5000 rpm to spin down undissolved cellular debris.
14. Approximately 250-300 μl supernatant was transferred into a 96 well microtitre plate.

### Target Amplification

The DNA from a P. hybrida EMS mutant population of ˜11,500 M2 individuals (2000 M1 families) was arrayed in 23 96-well microtitre plates with the DNA from up to 6 M2 siblings collected in each well (576 individuals per plate). For each plate an equimolar aliquot of DNA from each well was collected into a single 1.5 ml micro-centrifuge tube. This was done for each of the 23 plates. These were referred to as the plate pools and were used as DNA template for the following PCRs;

### PCR Primers:

| Primer Name | Primer Sequences |
|---|---|
| PhGene2 Forward | GAGGCTTTGCTGTTTGCTTC |
| | (SEQ ID NO: 1) |
| PhGene2 Reverse | CATGCAGAAACTCCCTATTCAGA |
| | (SEQ ID NO: 2) |
| PhGene1 Forward | CAAGAAGAAATGTCGAATGTTGTAG |
| | (SEQ ID NO: 3) |
| PhGene1 Reverse | GGTGACACACATCGCATCAA |
| | (SEQ ID NO: 4) |
| PhGene3 Forward | GACCATGGCTTTGTTACTGGATA |
| | (SEQ ID NO: 5) |
| PhGene3 Reverse | GAATCTGCGAGCTTCATAATACTTATCT |
| | (SEQ ID NO: 6) |

### PCR Conditions:

| Step | Description | PhGene1 | PhGene2 | PhGene3 |
|---|---|---|---|---|
| 1 | Incubate at 95° C. | 2 minutes | 2 minutes | 2 minutes |
| 2 | Incubate at 95° C. | 15 seconds | 15 seconds | 15 seconds |
| 3 | Incubate at 61.6° C. | 35 seconds | 20 seconds | 35 seconds |
| 4 | Incubate at 68° C. | 6 minutes | 1 minutes | 6 min 15 sec |
| | | 20 seconds | 10 seconds | |
| 5 | Cycle to step 2 for | 39 more times | 39 more | 39 more |
| | | | times | times |
| 6 | Incubate at 68° C. | 5 minutes | 5 minutes | 5 minutes |
| 7 | Incubate at 4° C. | Hold | Hold | Hold |

All PCR reactions were carried out in a solution of 10×PCR Buffer, 5 mM dNTPs, 25 mM MgCl2, 0.25 pmol/μl of forward primer, 0.25 pmol/μl of reverse primer, 10 Units Platinum Taq DNA polymerase (Life Technologies). Five replicates of each reaction were performed.

### Amplicon Pooling

The 5 PCR replicates for each amplicon were pooled into a single 1.5 ml micro-centrifuge tube. These were called amplicon pools. To confirm success of the PCRs 5 μl of each amplicon pool was run on a 2% agarose gel. If a band was weak or absent the 5 PCR replicates and pooling were done again. Equimolar amounts of 4 amplicon pools were combined to create 384-well amplicon pools. This was done to have all individuals represented on our 384-well HRM plates in single pools. Each 384-well amplicon pool was run through a QIAquick PCR Purification column (Qiagen) and the amount of DNA in each 384-well amplicon pool quantified using a fluorimeter and Horchst stain. All of the 384-well amplicon pools that used the same plate pool as DNA template for PCR were combined in equimolar amounts and then distributed to one of three library pools to be sequenced.

### Illumina Sequencing

Paired-end (PE) libraries were constructed for each of the three library pools using the Illumina TruSeq Sample Preparation Kit (Illumina) with barcoding. The average insert size for each library was ˜100 bp. The PE libraries where sequenced on an Illumina HisSeq 2000 instrument generating ˜200 million 100-bp PE reads. Library construction and sequencing were contracted out to the Plant Biotechnology Institute, National Research Council in Saskatoon, Saskatchewan.

### Sequence Processing

Data from our sequencing provider was delivered as 6 sequence files in FASTQ format, a forward and reverse sequence file for each of the library pools. PE reads were combined into a composite read using the software SHERA (Rodrigue et al, PLoS One 4:34761). The software cutadapt was used to remove primer, adapter and Illumina library barcodes from the composite creates (Martin, Bioinformatics in Action 17: 10-12). RepeatMasker was used to mask adapter and primer fusions in the composite reads that cutadapt could not process (Smit and Hubley RepeatModeler Open-1.0.). Following masking a stringent quality removal took place using custom programs written in perl. This is a two step process where all base calls in the composite read not supported by both high confidence PE reads (Phred quality score <60) are masked. Following masking the 5′ and 3′ strings of the masking character were removed. The resulting sequences were referred to as high quality (HQ) composite reads.

To create references sequences for read mapping the HQ composite reads were used for a de novo assembly using SOAPdenovo-Trans (http://soap.genomics.cn). For PhGene3 and PhGene2 full-length reference sequences were created of 6407 and 1261 by respectively while PhGene1 was separated into 2 contigs with a length totalling 6266 bases. The two PhGene1 contigs were unable to be fully assembled because of a highly heterozygous region of approximately 20 bases separating the two contigs. The two contigs were concatenated by a stretch of 100 ambiguity characters to serve as a single read mapping reference sequence. HQ composite reads were mapped to the three reference sequences using the software Bowtie2 (Langmeda and Salzberg, Nature Methods 9(4): 357-359). Bowtie2 was configured to allow for a single mismatch between reads and reference, for end-to-end mapping, and to not penalize for mapping masked bases. Using the software SAMtools (Li et al, Bioinformatics 25:2078-2079) and custom perl programs the occurrence of the 4 bases was tallied at each position of the alignment created by the mapping of HQ composite reads to the reference sequences.

### Statistical Analysis

At most positions of the read mapped reference sequences there were a limited number of occurrences of mapped non-reference bases. These variants can be from sequencing errors not corrected/masked by creating HQ composite reads, from errors introduced into the amplicons during PCR which were then sequenced, or they could be true incidents of mutation. The distribution of the log10 values of the non-reference base counts across all positions created normal distributions. Across all three reference sequences distributions of all possible transitions and transversions were constructed. To assign a probability of a non-reference base call to a position a z-score followed by a p-value were calculated using the distribution created for the base change of interest.

### Positive Controls

For the genes PhGene1 and PhGene2 13 mutations from the population were previously identified. These 13 mutations were used as positive controls to gauge the sensitivity of our new method. Using a method of an embodiment of the invention, the presence of 12 of these were verified at a p-value <0.01 (Table 1). The final positive control was found at a p-value of 0.05.

TABLE 1
Identification of Positive Controls.
In our petunia population we had previously identified 13 mutations.
We were able to verify the existence of these mutations using our
new method. Twelve of the 13 were found at a p-value <0.01.
Transitions and transversions were both detected.

| | | | | Nucleotide |
|---|---|---|---|---|
| Gene | Mutation | Effect | Probability | Position |
| PhGene1 | C->T | T58I | 4.31E−03 | 137 |
| PhGene1 | C->A | Silent | 2.41E−03 | 378 |
| PhGene1 | C->T | Intron | 3.04E−04 | 5890 |
| PhGene1 | G->A | Intron | 1.03E−03 | 5986 |
| PhGene1 | G->A | Intron | 8.42E−03 | 5990 |
| PhGene1 | G->A | P381S | 1.82E−03 | 6082 |
| PhGene2 | G->A | Silent | 5.01E−02 | 153 |
| PhGene2 | T->A | N90I | 3.44E−05 | 197 |
| PhGene2 | G->T | E74* | 2.31E−04 | 280 |
| PhGene2 | G->A | Silent | 2.17E−03 | 342 |
| PhGene2 | G->C | L191V | 1.56E−05 | 1045 |
| PhGene2 | C->T | P196L | 7.18E−03 | 1061 |
| PhGene2 | G->A | E210K | 1.43E−03 | 1102 |

### Mutant Identification

Variations from the reference found to created a truncated protein or mis-spliced mRNA were identified through bioinformatics analysis. Changes of interest with a p-value threshold of p<0.001 were selected for HRM analysis. Only a single mutation not previously identified in our population was found in PhGene1 that met our criteria. Primers flanking the mutation were created and tested against wild-type P. hybrida DNA. DNA from our mutant petunia population was screened with HRM analysis using a Lightscanner 384 instrument (Idaho Technology). A single well was found to generate a curve different from the wildtype profile, that is the single well was identified as containing the DNA from the mutant plant. Seeds from the plants from which the genomic DNA of this aberrant sample was extracted were planted. Leaf tissue was collected from these plants and genomic DNA extracted using a DNeasy Plant Mini Kit (Qiagen). An amplicon containing the region of the mutation was PCR amplified with the primers CTTTCTACTAGTTCACCTTACGAACA (forward; SEQ ID NO:7) and GGAACCTCTCATTTGTCAAGC (reverse; SEQ ID NO:8) with a standard PCR cocktail and 1× LCGreen HRM dye (Idaho Technology). The mutation confirmed through Sanger sequencing.

### Example 2

A second experiment was performed where 5 gene regions totalling ˜22,989 by were interrogated for mutations using our method. The method was carried out as set forth in FIGS. 2 to 4, and ˜200 million 100 by paired-end reads were generated on a single HiSeq 2000 lane. In these empirical data, much more noise than the simulation was encountered arising likely from polymerase errors from the PCR amplification stage of the process

### Gene Target Identification

Five gene targets were identified based on mutant phenotypes observed in Arabidopsis thaliana; PhGene4, PhGene5, PhGene6a, PhGene6b, PhGene6c. Reciprocal TBLASTN/BLASTP searches using the protein sequence of the A. thaliana genes against an in-house transcriptome database of Petunia hybrida identified putative P. hybrida orthologs of the A. thaliana targets.

### DNA Isolation

The DNA isolation protocol used was as described in Example 1.

### Target Amplification

The DNA from a P. hybrida EMS mutant population of ˜8,400 M2 individuals (1400 M1 families) was arrayed in 15 96-well microtitre plates with the DNA from up to 6 M2 siblings collected in each well (576 individuals per plate). Equimolar aliquots of DNA from the 15 96-well plates were arrayed into 4 384-well plates. For each of the four plates an equimolar aliquot of DNA from each well was collected into a single 1.5 ml micro-centrifuge tube. This was done for each of the plates for a total of 4 micro-centrifuge tubes each containing the DNA from three or four different 96-well microtitre plates. These are referred to as the plate pools and were used as DNA template for the following PCRs;

### PCR Primers:

| Primer Name | Primer Sequences |
|---|---|
| PhGene4 Forward | AAACCCTAGGGGAGAGAGACC |
| | (SEQ ID NO: 9) |
| PhGene4 Reverse | ATAATCCATTTGCACATTTGCTC |
| | (SEQ ID NO: 10) |
| PhGene5 Forward | CGAAGAAGGTCTGGCCTATTAAG |
| | (SEQ ID NO: 11) |
| PhGene5 Reverse | GGTCCTGAACAAGAAGATACCTACAC |
| | (SEQ ID NO: 12) |
| PhGene6a Forward | GGTGCTGCCAGTACTCAGG |
| | (SEQ ID NO: 13) |
| PhGene6a Reverse | CTGTTAGACCCACTTTGCAATTC |
| | (SEQ ID NO: 14) |
| PhGene6b Forward | CGCCGTTACTCAAGTGGTG |
| | (SEQ ID NO: 15) |
| PhGene6b Reverse | TGACTTTGTTCAACGCTTTGTC |
| | (SEQ ID NO: 16) |
| PhGene6c Forward | TTAGGTGTTACAGGGATAATAAGCAGT |
| | (SEQ ID NO: 17) |
| PhGene6c Reverse | CAAGAATCTAGTGACCCATTTGC |
| | (SEQ ID NO: 18) |

| Step | Description | PhGene1 | PhGene2 | PhGene3 |
|---|---|---|---|---|
| 1 | Incubate at 95° C. | 2 minutes | 2 minutes | 2 minutes |
| 2 | Incubate at 95° C. | 15 seconds | 15 seconds | 15 seconds |
| 3 | Incubate at 61.6° C. | 35 seconds | 20 seconds | 35 seconds |
| 4 | Incubate at 68° C. | 6 minutes | 1 minutes | 6 min; |
| | | 20 seconds | 10 seconds | 15 sec |
| 5 | Cycle to step 2 for | 39 more times | 39 more | 39 more |
| | | | times | times |
| 6 | Incubate at 68° C. | 5 minutes | 5 minutes | 5 minutes |
| 7 | Incubate at 4° C. | Hold | Hold | hold |

### PCR Conditions:

All PCR reactions were carried out in a solution of 10×PCR Buffer, 5 mM dNTPs, 25 mM MgCl2, 0.25 pmol/μl of forward primer, 0.25 pmol/μl of reverse primer, 10 Units Platinum Taq DNA polymerase (Life Technologies). Five replicates of each reaction were performed.

### Amplicon Pooling

The 12 PCR replicates for each amplicon were pooled into a single 1.5 ml micro-centrifuge tube. These were called amplicon pools. To confirm success of the PCRs 5 μl of each amplicon pool was run on a 2% agarose gel. If a band was weak or absent the 12 PCR replicates and pooling were done again. Equimolar amounts of 4 amplicon pools were combined to create 384-well amplicon pools. This was done to have all individuals represented on our 384-well HRM plates in single pools. Each 384-well amplicon pool was run through a QIAquick PCR Purification column (Qiagen) and the amount of DNA in each 384-well amplicon pool quantified using a fluorimeter and Horchst stain. All of the 384-well amplicon pools that used the same plate pool as DNA template for PCR were combined in equimolar amounts and then distributed to one of four library pools to be sequenced.

### Illumina Sequencing

Paired-end (PE) libraries were constructed for each of the four library pools using the Illumina TruSeq Sample Preparation Kit (Illumina) with barcoding. The average insert size for each library was ˜100 bp. The PE libraries where sequenced on an Illumina HiSeq 2000 instrument generating ˜200 million 100-bp PE reads. Library construction was contracted out to the Farncombe Metagenomics Facility, McMaster University, Hamilton, Ontario, Canada and sequencing was contracted out to the Genome Quebec and McGill University Innovation Centre, Montreal, Quebec, Canada.

### Sequence Processing

Data from our sequencing provider was delivered as 8 sequence files in FASTQ format, a forward and reverse sequence file for each of the library pools. PE reads were combined into a composite read using the software SHERA (Rodrigue et al, PLoS One 4:34761). The software cutadapt was used to remove primer, adapter and Illumina library barcodes from the composite creates (Martin, Bioinformatics in Action 17: 10-12). RepeatMasker was used to mask adapter and primer fusions in the composite reads that cutadapt could not process (Smit and Hubley RepeatModeler Open-1.0.). Following masking a stringent quality removal took place using custom programs written in perl. This is a two step process where all base calls in the composite read not supported by both high confidence PE reads (Phred quality score<60) are masked. Following masking the 5′ and 3′ strings of the masking character were removed. The resulting sequences were referred to as high quality (HQ) composite reads.

To create references sequences for read mapping the HQ composite reads were used for a de novo assembly using SOAPdenovo-Trans (http://soap.genomics.cn). HQ composite reads were then mapped to the five reference sequences using the software Bowtie2 (Langmeda and Salzberg, Nature Methods 9(4): 357-359). Bowtie2 was configures to allow for a single mismatch between reads and reference, for end-to-end mapping, and to not penalize for mapping masked bases. Using the software SAMtools (Li et al, Bioinformatics 25:2078-2079) and custom perl programs the occurrence of the 4 bases was tallied at each position of the alignment created by the mapping of HQ composite reads to the reference sequences.

### Statistical Analysis

At most positions of the read mapped reference sequences there were a limited number of occurrences of mapped non-reference bases. These variants can be from sequencing errors not corrected/masked by creating HQ composite reads, from errors introduced into the amplicons during PCR which were then sequenced, or they could be true incidents of mutation. The distribution of the log10 values of the non-reference base counts across all positions created normal distributions. Across all five reference sequences distributions of all possible transitions and transversions were constructed. To assign a probability of a non-reference base call to a position a z-score followed by a p-value were calculated using the distribution created for the base change of interest.

### Mutant Identification

Variations from the reference found to created a truncated protein, mis-spliced mRNA or detrimental changes as determined by the software SIFT (Ng adn Henikoff, Nucleic Acids Res. 1; 31(13):3812-4) were identified through bioinformatics analysis. Changes of interest with a p-value threshold of p<0.001 were selected for HRM analysis. Primers flanking the mutations were created and tested against wild-type P. hybrida DNA. DNA from our mutant petunia population was screened with HRM analysis using a Lightscanner 384 instrument (Idaho Technology). For 10 of 14 mutations of interest identified through bioinformatics analysis a single well was found to generate a curve different from the wildtype profile, that is the single well was identified as containing the DNA from the mutant plant. Seeds from these plants from which the genomic DNA of this aberrant sample was extracted were planted. Leaf tissue was collected from these plants and genomic DNA extracted using a DNeasy Plant Mini Kit (Qiagen). DNA from individual plants was subject to the same HRM conditions as the 384-well pool. Each of the 10 positives HRM signals repeated in individual plants and the mutation confirming with Sanger sequencing.

### Example 3

A third experiment was performed where 6 gene regions totalling 30,563 by were interrogated for mutations using our method. The method was carried out as set forth in FIGS. 2 to 4, and ˜33 million 250 by paired-end reads were generated from two runs on an Illumina MiSeq using version 2 500 cycle sequencing kits.

### Gene Target Identification

Six gene targets were identified based on mutant phenotypes observed in Arabidopsis thaliana; PhGene7, PhGene8, PhGene9, PhGene10a, PhGene10b, PhGene10c. Reciprocal TBLASTN/BLASTP searches using the protein sequence of the A. thaliana genes against an in-house transcriptome database of Petunia hybrida identified putative P. hybrida orthologs of the A. thaliana targets.

### DNA Isolation

The DNA isolation protocol used was as described in Example 1.

### Target Amplification

| Primer Name | Primer Sequences |
|---|---|
| PhGene7 Forward | CAAGAAGAAATGTCGAATGTTGTAG (SEQ ID NO: 19) |
| PhGene7 Reverse | GGTGACACACATCGCATCAA (SEQ ID NO: 20) |
| PhGene8 Forward | GAGGCTTTGCTGTTTGCTTC (SEQ ID NO: 21) |
| PhGene8 Reverse | CATGCAGAAACTCCCTATTCAGA (SEQ ID NO: 22) |
| PhGene9Forward | CGACGGCGGAGATATAATTAAC (SEQ ID NO: 23) |
| PhGene9Reverse | ATAATCCATTTGCACATTTGCTC (SEQ ID NO: 24) |
| PhGene10Forward | CCAGGACACTCTTTCTAGTGTTGA (SEQ ID NO: 25) |
| PhGene10Reverse | GGTCCTGAACAAGAAGATACCTACAC (SEQ ID NO: 26) |
| PhGene11a Forward | TTGGTGTTTCTGCAGGCTTAATA (SEQ ID NO: 27) |
| PhGene11a Reverse | CTGTTAGACCCACTTTGCAATTC (SEQ ID NO: 28) |
| PhGebe11b Forward | CGCCGTTACTCAAGTGGTG (SEQ ID NO: 29) |
| PhGene11b Reverse | TGACTTTGTTCAACGCTTTGTC (SEQ ID NO: 30) |
| PhGene11c Forward | TTAGGTGTTACAGGGATAATAAGCAGT (SEQ ID NO: 31) |
| PhGene11c Reverse | CAAGAATCTAGTGACCCATTTGC (SEQ ID NO: 32) |

The DNA from a P. hybrida EMS mutant population of ˜6,600 M2 individuals (1100 M1 families) was arrayed in 12 96-well microtitre plates with the DNA from up to 6 M2 siblings collected in each well (576 individuals per plate). Equimolar aliquots of DNA from the 12 96-well plates were arrayed into 3 384-well plates For each of the 3 plates an equimolar aliquot of DNA from each well was collected into a single 1.5 ml micro-centrifuge tube. This was done for each of the plates for a total of 3 micro-centrifuge tubes each containing the DNA from four different 96-well microtitre plates. These are referred to as the plate pools and were used as DNA template for the following PCR reactions:

### PCR Primers:

### PCR Conditions:

| Step | Description | PhGene1 | PhGene2 | PhGene3 |
|---|---|---|---|---|
| 1 | Incubate at 95° C. | 2 minutes | 2 minutes | 2 minutes |
| 2 | Incubate at 95° C. | 15 seconds | 15 seconds | 15 seconds |
| 3 | Incubate at 61.6° C. | 35 seconds | 20 seconds | 35 seconds |
| 4 | Incubate at 68° C. | 6 minutes | 1 minutes | 6 min, |
| | | 20 seconds | 10 seconds | 15 sec |
| 5 | Cycle to step 2 for | 39 more times | 39 more | 39 more |
| | | | times | times |
| 6 | Incubate at 68° C. | 5 minutes | 5 minutes | 5 minutes |
| 7 | Incubate at 4° C. | Hold | Hold | Hold |

All PCRs were carried out in a solution of 10×PCR Buffer, 5 mM dNTPs, 25 mM MgCl2, 0.25 pmol/μl of forward primer, 0.25 pmol/μl of reverse primer, 10 Units Platinum Taq DNA polymerase (Life Technologies). Five replicates of each reaction were performed.

### Amplicon Pooling

The 12 PCR replicates for each amplicon were pooled into a single 1.5 ml micro-centrifuge tube. These were called amplicon pools. To confirm success of the PCRs 5 μl of each amplicon pool was run on a 2% agarose gel. If a band was weak or absent the 12 PCR replicates and pooling were done again. Each 384-well amplicon pool was run through a QIAquick PCR Purification column (Qiagen) and the amount of DNA in each 384-well amplicon pool quantified using a fluorimeter and Horchst stain. All of the 384-well amplicon pools that used the same plate pool as DNA template for PCR were combined in equimolar amounts and then distributed to one of three library pools to be sequenced.

### Illumina Sequencing

Paired-end (PE) libraries were constructed for each of the three library pools using the Illumina TruSeq Sample Preparation Kit (Illumina) with barcoding. The average insert size for each library was ˜250 bp. The PE libraries where sequenced on an Illumina MiSeq instrument generating ˜33 million 250-bp PE reads. Library construction was contracted out to the Farncombe Metagenomics Facility, McMaster University, Hamilton, Ontario, Canada.

### Sequence Processing

Data from our sequencing provider was delivered as 6 sequence files in FASTQ format, a forward and reverse sequence file for each of the library pools. PE reads were combined into a composite read using the software SHERA (Rodrigue et al, PLoS One 4:34761). The software cutadapt was used to remove primer, adapter and Illumina library barcodes from the composite creates (Martin, Bioinformatics in Action 17: 10-12). RepeatMasker was used to mask adapter and primer fusions in the composite reads that cutadapt could not process (Smit and Hubley RepeatModeler Open-1.0.). Following masking a stringent quality removal took place using custom programs written in perl. This is a two step process where all base calls in the composite read not supported by both high confidence PE reads (Phred quality score<60) are masked. Following masking the 5′ and 3′ strings of the masking character were removed. The resulting sequences were referred to as high quality (HQ) composite reads.

To create references sequences for read mapping the HQ composite reads were used for a de novo assembly using SOAPdenovo-Trans (http://soap.genomics.cn). HQ composite reads were then mapped to the five reference sequences using the software Bowtie2 (Langmeda and Salzberg, Nature Methods 9(4): 357-359). Bowtie2 was configures to allow for a single mismatch between reads and reference, for end-to-end mapping, and to not penalize for mapping masked bases. Using the software SAMtools (Li et al, Bioinformatics 25:2078-2079) and custom perl programs the occurrence of the 4 bases was tallied at each position of the alignment created by the mapping of HQ composite reads to the reference sequences.

### Statistical Analysis

At most positions of the read mapped reference sequences there were a limited number of occurrences of mapped non-reference bases. These variants can be from sequencing errors not corrected/masked by creating HQ composite reads, from errors introduced into the amplicons during PCR which were then sequenced, or they could be true incidents of mutation. The distribution of the log10 values of the non-reference base counts across all positions created normal distributions. Across all five reference sequences distributions of all possible transitions and transversions were constructed. To assign a probability of a non-reference base call to a position a z-score followed by a p-value were calculated using the distribution created for the base change of interest.

### Mutant Identification

Variations from the reference found to created a truncated protein, mis-spliced mRNA or detrimental changes as determined by the software SIFT (Ng adn Henikoff, Nucleic Acids Res. 1; 31(13):3812-4) were identified through bioinformatics analysis. Changes of interest with a p-value threshold of p<0.001 were selected for HRM analysis. Primers flanking the mutations were created and tested against wild-type P. hybrida DNA. DNA from our mutant petunia population was screened with HRM analysis using a Lightscanner 384 instrument (Idaho Technology). For 27 of 37 mutations of interest identified through bioinformatics analysis, a single well was found to generate a curve different from the wildtype profile. Seeds from these plants from which the genomic DNA of this aberrant sample was extracted were planted. Leaf tissue was collected from these plants and genomic DNA extracted using a DNeasy Plant Mini Kit (Qiagen). DNA from individual plants was subject to the same HRM conditions as the 384-well pool. Mutations for each of the 27 positives HRM signals were confirmed with Sanger sequencing.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method for isolation of a member of a population which has one or more mutation(s) in one or more target sequence(s) in a population, comprising the steps of:
(a) pooling genomic DNA isolated from each member of said population;
(b) amplifying the one or more target sequence(s) in the pooled genomic DNA;
(c) pooling the amplification products of step (b) to create a library of amplification products;
(d) sequencing the amplified products by pair-end sequencing to produce paired-end reads for each sequencing reaction or obtaining paired-end sequence reads for the amplified products;
(e) merging the paired-end reads into composite read(s);
(f) mapping the composite read(s) to reference sequence(s) to identify mutation(s) in the one or more target sequence(s); and
(g) identifying member(s) of the population comprising one or more of the identified mutations in the target sequence(s).

2. The method of claim 1, wherein said population is a mutagenized population.

3. The method of claim 2, wherein said population is mutagenized by mutation-inducing chemicals, ionizing radiation, targeted nucleotide exchange or region targeted mutagenesis.

4. The method of claim 1, wherein said member(s) of the population comprising one or more of the identified mutations in the target sequence(s) are identified by high-resolution DNA melting (HRM).

5. A method for identifying one or more mutation(s) in one or more target sequence(s) in a population, comprising the steps of:
(a) pooling genomic DNA isolated from each member of said population;
(b) amplifying the one or more target sequence(s) in the pooled genomic DNA;
(c) pooling the amplification products of step (b) to create a library of amplification products;
(d) sequencing the amplified products by pair-end sequencing to produce paired-end reads for each sequencing reaction or obtaining paired-end sequence reads for the amplified products;
(e) merging the paired-end reads into composite read(s); and
(f) mapping the composite read(s) to reference sequence(s) to identify mutation(s) in the target sequence(s).


SEQUENCE LISTING (35 sequences)

SEQ ID NO: 1 - DNA, 20 bp, Artificial Sequence
  primer
gaggctttgctgtttgcttc

SEQ ID NO: 2 - DNA, 23 bp, Artificial Sequence
  primer
catgcagaaactccctattcaga

SEQ ID NO: 3 - DNA, 25 bp, Artificial Sequence
  primer
caagaagaaatgtcgaatgttgtag

SEQ ID NO: 4 - DNA, 20 bp, Artificial Sequence
  primer
ggtgacacacatcgcatcaa

SEQ ID NO: 5 - DNA, 23 bp, Artificial Sequence
  primer
gaccatggctttgttactggata

SEQ ID NO: 6 - DNA, 28 bp, Artificial Sequence
  primer
gaatctgcgagcttcataatacttatct

SEQ ID NO: 7 - DNA, 26 bp, Artificial Sequence
  primer
ctttctactagttcaccttacgaaca

SEQ ID NO: 8 - DNA, 21 bp, Artificial Sequence
  primer
ggaacctctcatttgtcaagc

SEQ ID NO: 9 - DNA, 21 bp, Artificial Sequence
  primer
aaaccctaggggagagagacc

SEQ ID NO: 10 - DNA, 23 bp, Artificial Sequence
  primer
ataatccatttgcacatttgctc

SEQ ID NO: 11 - DNA, 23 bp, Artificial Sequence
  primer
cgaagaaggtctggcctattaag

SEQ ID NO: 12 - DNA, 26 bp, Artificial Sequence
  primer
ggtcctgaacaagaagatacctacac

SEQ ID NO: 13 - DNA, 19 bp, Artificial Sequence
  primer
ggtgctgccagtactcagg

SEQ ID NO: 14 - DNA, 23 bp, Artificial Sequence
  primer
ctgttagacccactttgcaattc

SEQ ID NO: 15 - DNA, 19 bp, Artificial Sequence
  primer
cgccgttactcaagtggtg

SEQ ID NO: 16 - DNA, 22 bp, Artificial Sequence
  primer
tgactttgttcaacgctttgtc

SEQ ID NO: 17 - DNA, 27 bp, Artificial Sequence
  primer
ttaggtgttacagggataataagcagt

SEQ ID NO: 18 - DNA, 23 bp, Artificial Sequence
  primer
caagaatctagtgacccatttgc

SEQ ID NO: 19 - DNA, 25 bp, Artificial Sequence
  primer
caagaagaaatgtcgaatgttgtag

SEQ ID NO: 20 - DNA, 20 bp, Artificial Sequence
  primer
ggtgacacacatcgcatcaa

SEQ ID NO: 21 - DNA, 20 bp, Artificial Sequence
  primer
gaggctttgctgtttgcttc

SEQ ID NO: 22 - DNA, 23 bp, Artificial Sequence
  primer
catgcagaaactccctattcaga

SEQ ID NO: 23 - DNA, 22 bp, Artificial Sequence
  primer
cgacggcggagatataattaac

SEQ ID NO: 24 - DNA, 23 bp, Artificial Sequence
  primer
ataatccatttgcacatttgctc

SEQ ID NO: 25 - DNA, 24 bp, Artificial Sequence
  primer
ccaggacactctttctagtgttga

SEQ ID NO: 26 - DNA, 26 bp, Artificial Sequence
  primer
ggtcctgaacaagaagatacctacac

SEQ ID NO: 27 - DNA, 23 bp, Artificial Sequence
  primer
ttggtgtttctgcaggcttaata

SEQ ID NO: 28 - DNA, 23 bp, Artificial Sequence
  primer
ctgttagacccactttgcaattc

SEQ ID NO: 29 - DNA, 19 bp, Artificial Sequence
  primer
cgccgttactcaagtggtg

SEQ ID NO: 30 - DNA, 22 bp, Artificial Sequence
  primer
tgactttgttcaacgctttgtc

SEQ ID NO: 31 - DNA, 27 bp, Artificial Sequence
  primer
ttaggtgttacagggataataagcagt

SEQ ID NO: 32 - DNA, 23 bp, Artificial Sequence
  primer
caagaatctagtgacccatttgc

SEQ ID NO: 33 - DNA, 6320 bp, Artificial Sequence
  PhGene1-partial
cttatcaaggccggcatcggtggcgaaagagacccttgtgctattgtccccaacaccatggcccgtcccctttcttccaagaaatggcttttagcagatcagcttctttcccctgacgtggacctcacttccgccaccctccgccgccctttcgaccggggctaccttatcaaccccgaccttcagtcttccctctggtcccacgtcttctccaacctcctcaaaatcactccttcacactcctccctgctcctcactgaaccccttttcaatctcccttccattcaacgctccgttgatgaaatcgttttcgaagacttcaatttcaaggccctttacgtttctgattccccttccttggtccatctttatgaggcctctcgtcgcccttatggccttgtctctaaagcccagtgcagcttagttgtcgactctggattctcgtttactcatgcctcccccgtgtttcagaattttaccttgaattatggggtgaagaggcttgatcttggtggcaaagcattgactaattacttgaaggagttggtgagttatagaagtgttaatttgatggatgaaaccttccttatggatgatgttaaggagaaactttgctttgtctcgttggacgttcccagggacttgcacattgctaggtatattgctccttctcttctagctttttcccgttccaatttatgtttgattgagcccggagtttaacaaagaaaagactttacatacttgtgatcttaaatatatcataacatttgcgtggctatgatagcttgtcattttaagggtaaaatagcaagtgtaagttaaattgtttttgttacaaaatgtgcatagacacattatcatatagtatgcattatacattaataggtagagatatttgattatatttccttagacagattcctatgttactcggactcttcactttcggtgccgcacccatgtcgactgggtgtgggcgtgggatctgtacccgatttggtcaaatgattttagatgctttgaccaaaatcgacggagaaattctggagagattcaatgatttctctaatcaaaacaaaagtaagggaaaattgaacgaaatagagtaccttgtattattgtatatagaaatttttatgtcactccttttccttttatctccttcaggaattctcctattgatcaatattttttcctcaggtttttcacataatatctcataatttaggttttagaactctactttcatatatttgaattttttttagccgaatccctgcacctgtatccataactggatccgtatccccaaatcttaaaatttaaatcaggatccgatctctagatccgcacccgtatcgaacacccgcacccgagtctgagtaacttagacagattcacttgtacatgtatacatagctctcgcctctcattcttggagcattaatatagaaaatttccttagatagttagggatgtgacaacagagtgaatgaagatatgattgctactttctacctcaatttcacacaagtagcatttgttcactagctgtaaacctcttctttgtaacttatcttgagttagacacactttcctattcaaaacaaataactttgctaggtgcttagtgttcttgacatgtttttgggaccttgtgttagctgatgtgcattagattgtaacaggtcataacgagatttaatagtgattatacccttttttgtgctttccatactagactactagttgatcatccctgctggatagtgcttcaaaagaaactaatatgttgataatttctgtatttcctccaagctcgcaatcatttaggtttcttctcaaacttaagtttgagcagctgtcaaccctgcatgcttctattgtgatatcactattttggaccagggtgtaaatagaagggaattgctcatgaagaggagggtgtcccatccatttatcttgccaaaaagctatttttcttccattgattattttgaaatcagtgtgctgcaaaaacaaaggccacaaggttctgatgtgtctccaatctccatgcacaaaaccgtcgaggtaaagaattaacccattcgttatgatcacattcctccataatacattagtttctacatatgattctccatagctatttcattagaagactcctattttgcagtattagattcttaatgcccaagccaccatttttcctttcttgtaatgtcgttgctccatttaaccaagttgaacgttttggcctccttatttccttatcataaaatgtctcttctcactttatccattcttttttctaccgtgagaggcaaagtaaataatgacatatgttggaatggcatcaagaacactatgcataaatcaatcttccgcctagggataggtgtgatgcttcttctgattggataggcttcattcacatagttccaccaccttttgccaattgaagtcagatctactctttgctcccaaaggcatcccaaggtaagctgtgggaagagtttctatttggcatcccaagatggtggctaggctttgtaagtcttgaactaagttgacaggaatcaatttactctttgaccaattttcataaagtcttgacacaacttcaaatacattgtgtcaatctcaaatgtcttgatttgtctaccgccggctcataagaaacaagtgtgtcatcatcatacagtaaatgtgaaatggccagaacatcttgccctctgatcattttgaaatccttgatcctcaatccttcattcactgccaagtgccaagttaattgttttgctgagccttccataagtaacaagaactagaaaggaaatactaggtctccttgtcttagccccccttgtgtcgggaaaacaccttctggagaacctatgattagcacagaaaattgaactgtgcagatacaaaaccaaatcctttgttgcctttttcttccaaaaccctaatcctttagcatctttagtggaagattccagtttatgtgatcataagtcttttcaatgtctagcttactcatgatccctggttttttctcctttctttaattgaggcattcatttgcttttaacacaacatctgttatttctcttccgttcaggaatggatggaaacagaaacaagaaccattttatttaatagggattcaattttgaaagataaaaaggttgaaggcttcaggttagggacctgaaaatgcgtaattagagtctacttaacaaatgattatggaggttcaccaaaagaagaaggttcactatggagaaatgtcattagagccaagtgtggcctgtatagtaagtggataactaagatggctattttttcctgatgtacagtctctttggaaaagcataggattctttgggatgatttttttgggcaaaaaaatatttcttgttaggagatggaaggatgattagcttttggcatggctcttgtataggtcacactattggactaagtaaattaatacttgggatgaatctattatttatttgttttatttttaaatgcccccttaaaccagattcatcactccacccaatttttgaagatatcaatctttactggctactaaagatatccacaacttggtcttgtgacttcacaagctcgaggtcaaattctttctctaccttgtccatatgtacaatctctttgatgtgagcgacatttttcatctttcacatttagaaaaacctttgcccgtaagtttgttcaaatgattcttatttcgacttttgtaaactttcttcagatacttctgaagatcttgcaagttcatccatgatcatggtgtccaaatctttaggctcttcaatgacaacaacaacacgattaaatagtgttcagggatcgtggaattcttgcaacggcctgatcatctctcaaatcttcttcatatctcttcatcatcttcattgtcgtcgtcatcttcatcttttttcgggtttcatggagaaaaagtttgatgagagtgaaaattgtcttattcaatctttcatggcagcagcctcattctctatcttcctattcaagattcatcgtcatgcagctactccttgagcaacttattcatcagggttgacgctcttattgggttccttgctcttaaaaacaatccttttaacttatgaaatctttgtagtcacctgctaaaatacattaatctgatgcttcccctagtccagcattctcataactataactgctaatctgtctgacattcattttgttctgaagattcattcttttgaatcttaaaattctgatacttcacaggtccttcaagacttgggctttgataccaaatgttggaaatttagaactgtactctcagatcactcaagttctcttgaaataaatgatacttctttattctcgctacttgcttacattaaatggctatagcgcgtagatatttttaaataaatttctatcatgaactagtcagtatatgtatcttgatttggttcgaaacgtttttcatgaataagttatacctgcatattagtattactttcctaatacatgaattacgtaaattcatgtactacagatgaatctactttatattttcaacagacacctcgcaagtggagttatcctgatcttttcactctggcagccaaccaagaagacctcagtggcagaaaactgctcaactatggttggcatattatttttagttgacattgcaatgattggaagtagacaaaacatgtaatctacttcaacaactaggaactcaaggtttatccttagataagatggacactattggtcataaaggcattgttgtatagaagattgggtagcatttatagaaaaccttatgtttttgtaggtggtcggtttttcttttttgggtatactaatatactactaagccagcagaaaatccacattaaagtcagatgctctgttgtgtttcctacatgattgggtctttcctgttcataacgactgcatatcttccaggaaaccaggaagggacaatttgtttaggtgcacatatgtgcttcctgatggtattacacataggaagggttttctgaaagatcctgaagaaccgaagagatacctatctctgtatgataaagctcaacagcaagtagcagcagaacaaaatgatgtggatcagcttgacaatgctgataattctgaggacaggagcaggagcaatgcatcaagaattgatttgacaaaaaatgtactctttccgatcccttattagttgcccccccccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttctgcgaatgaaagacacaagcggtggcttcaggtatgatgctttctactagttcaccttacgaacaggaatttggcttgacaaatgagaggttcctcgtcccggagatgatgtttcgtccagctgatttgggtctgtgtcaagacttatcttggaattctagttcagcctttgtatttaattcagcagtttccttcatttattcagcgtgttacaaatgattgagatttagacttcatctcaagctactttttttcctttcatgtgaactggagtgtccagaagtgtggccctatgaattcgtttattgccaagtacgaatttgatagttagcttctcagttattggtgatttacaggactgaatcaggctggacttgcagaatgcattgttcgagctgtcagttcgtgtcattctcatcttcaccctattctctatgagaggtagcgccgagcttctggaactttaaaagttatttagtaaaaatactttatgcttatcttccattctctaatgtaaattcattctgaaattgaacttggaagcatcattttgacgggaggcagcactctctttcctcattttgctgaaagactgtaagattcagcctcttttgcccattggaagaactctatctggatgtcctacttgcttacacagtcttgaaacttcatctaaatgcagagaaatggaccttcggccacttgttccagatgaatatcgtttaaagattacgattcaagaagagtatgtaccatccttttggagaaatttaaaccgataaagtggttctacttctctaacttgtcaacttttgttgcttatgcagtcctatactaggtgtttggcgtggagggtcacttttggcatcaagtcctgattttgatgcgatgtgtgtcacctcatgcagaaactccctattcagaagcaaacagcaaa

SEQ ID NO: 34 - DNA, 6407 bp, Artificial Sequence
  PhGene3 reverse complement
gaccatggctttgttactggataattcattcgatcgctttgctcggagaatccattgatgccaaactggaaaatgatgcaattgactttctcaaccgttgccaggtctgttcattttggattctgctatttaaactattaaatatatctgccaatcaagagtatggatgggcttggccccgataaaaatcaagcaagttagattattttatttacagccatagaattataaattattggaataaattctgatcacttgtgttgcaactatcaatcttgactcttgagtaaaatggactttagaagtactgctagtttttagaataccctcttagattgtaggttggtcaatctgtctttccggagtactggtatacagtatacctgtccttttttgctctttctgcccgcaattccactcatttgttttgagacttctcacacaataaaatgctactcctataccatttagtttcatgagttgtttgccgcctcgcaggataaaaatggtggctatggtggtggacctggtcaggcaagtcaaattctcctgctgcactcagttccactaggacagatttgattcagctgctttttacttcttttctcgttgagtttgattttgtaatgcctccatttgtttctggtatgcttgagtgtccctgagcgattattaattcgtattttgcttatcgtacctttaaagtttgacacgcccgaattatggctcatgttagtttcatcctctgaagattatttgacttggacacacacgtttgatttggataccctaaatttatgagtcaaatataattctattaaagtacttataataaaaaacaattctattagagggaaatagtcatccgaattgatagatatgccttcaattattgaccttgttcctcgttaatttctaagtcagcttgaaactcttttttcttttagagtcacaaatctacagttacaagcactctttagtccatttaatctgcttatttgtacctcctatcgttaaggctctttgaactaatacttgtattatattgctttgttcagatgcctcatcttgcaactacttatgctgcagtcaattcgcttataactttgggcagccgtaatgctctgtcatcaatcaataggtgcacagctattctatgttcttactggcactttatctatcatccactcttgctgacattgatctactaatatgcctcagagaaaaattgtatgcattttttctgcgaatgaaagacacaagcggtggcttcaggtatgatgctttctacttgtttaccttatttgatctgtccgccaatcatgtcttccttatctctttcctagtctactgtatctcctgttcgtcttctgattttaatcctacctgctaaaaagattattctgttttcactcctgaggggaactaggattttggatgaagacgcttttaccctgatcagaaaataaagcatctgagaacataaatgtataaagagtatgggttttctccagggggtggggggacattattgaggctgtatatccaaatggttttaacaaattctgatcagacgatgagcgcttgtctctaacattctgtttcagttttcagattctgttcaataataacttaccaatttccatctttggtaatgttatacttcaaatattctctgcaggatgcatgatggtggagaagtagatgttcgtgcctgctatactgctatttctgtaagtgtgatcctttgtaacactgtgctggcttgttctataatttctgataatatcgtcatcatttttattctgcttagatagaaaaacctgagataagcttaagagaaaatatgaaaattttgatcctgatttacttgatgaatcatttaagttacagatgttggaaaatttatgtaaaattcacagtccccctagcaacattcaagccatacctcgtctactcgtactgaagataattggatgaagtactctcgctgaatatcttcttgtgcttatcgaggatcaatttcttcaaacacgacttagtttttgaaaacataatttagattcaaatgtctccaaagtttcatctgaaaggcaatcaaagcctccagatgcatggctctcttgctatttaatagtgaatacttgattgcatacaccgttcttgcatgttacattcttaacttttgtcttttttctttcttagttatcttcggaataatgtgccttcgtgctaattgatatattagtttttcaggttgctagtatattgaacattgtggatgataagctgattcatggtgttggaaattacatcctaaggttggtctaagcagcaccctactactgctcatattctactttatctttacttctacggtgtgtatatatatatatataaagtttcaatttcttgatttagtgatggctgttgtcttccaagggaatgacacgatagataatggtgttacgatagaactaaaaaggatggctgaaataggggagtgcttaaggagtgcaatctgaaaaaaaacataccctttaaaatgaaagacaagttgtatatacaatagttgtgggattgatgctagttgatttgattcatattaactgatgttagttgtcagtgcatggtgattcgatcccaatggtattagaagcaaattatgatgccatttgcatgttcataactctgttctcactgcaagtagtagttgatgacagctttattaaaaacagtagcagttgatacttgatagacagctgccattcgttcgggattaaaatatgagatatgtatgatatttcctaattatttgcatcttggaaagtctacattgaaaaaaaataatgaaggaaatattctctattctagtttgttctcattcttgttttctcgagtttttggtaagagggatttattcttagttattgtcagatgtacaagataagaggagtggggaagctctttcttttccttaatgccctcagttttgttttgttcaaattgatcataataatatatttgctaggatatgcctttcctaagctgttctctcatatttgcttcagttgtcagacttatgaaggtggcattgctggcgagccaggtgctgaagctcatggtgggtaagtctcacctgggagatgtgcttatatttttctgctcattattctctttcactcgttaattgtcatagttagactatggcatctcaacttatgcgggtgagcccttcatgtatcattcattttctaggtacactttctgtggtttggccgcaatgattctgatcaatgaagtgaaccgattggacttgccaagattaattgtaagttgcttttgttgctttagaaggtgttagggaccgatgaataatcatattatgcatcttcccttgttttcgaactgatatataggcatgtagcattcctggcactgtcatgaaatggaccattagtgtattattttgttacaggccttcgtgactgatcaaatgaaactgagtccatagaagtgttgtaaaatggctgtcattttattcatcttcacaagtgttgtacattacatttttattacttctcatcaaaacacagggagaatgaaaatattgttaagatgagctaactctcagctgacaattgaagaagtggaagtattttaatgctatctcttctgagttcaggagttcattcaagtagttaagatggaagaagccactacaattaactcagtttttggttttgaaacggttgcctataattactaatatttgatcagtgaaatggtaggttattatgttttaacagatgcagctagttattggatatttaggaaccaaaagtaaagtagaactgaaagaagtgtgatgtattgaaagtaaacatatggaaaaaagtgaaatagatgttgaacagctggaataaattgcagagtttaggtggttgtccctcattagggaccctgtagtatgagacttttgagtgctttttataccacaatatggcaggaaggaagacatccaggaacccagaactgatcttataaaaaaataaatccaagtacccggaactggttggttttgaccttggtttgaccttctatttacttgagcagtccattattaagcagctgcactgggacccctttaccaggtttttctgctgcatcctggctggttggggttggattattaaccaaaagtattttgttccttttccgtctgtctgtctttcatatctatgtgactttctctgtctcttcttttagtagttttcttcagtttcttttctatgactaattcccatgaaattttatctacttcttgatgaagtgaaatcacttcagtttggtttgtatgatgatactgatgggtatgaaatgtggtgtgattatgtatcttaggattgggtggtatttagacaaggagtcgaaggtggatttcaaggcaggacaaataaattagttgatggctgctattccttttggcaggtggctatcagttttacttcatgtcctgaactctttcattttcagttttaactattattgagctaaagatgactttgattgaccagggtgcagtagcttttatgatacaaagattaaaattgatagtccatgaacaactagggctgtcaaatgagcttggtacagaaagttctgatgattcttcagcatcagggttatctgacgaagaagagcatttggaagggacatcctctcatgttcaggagacttcccctcttagagaagaaggtacatcaatttgaggaacagtaactcaaaggccaaaaaacgacatactcctgtatgattagctggtctaccctatgttgcatccataaagtccttggaatctgaatttaaaaatggatatcaagtgattagagaaatccagtggaattgggtgaaaagaatatattccattttaaactgcttcgcttgtcacaccctttgcatttgcgttgaaaataaaattgaatgggaaacctatagtgctatcagctggacttctccttaactaggtaaaaggacagcttggtgcacacagcatcccgcgttcactcagggtcggggccgcacctcaagggtgtgatgtacacagcctaccctgatgccctgatgcaagcatcagtggctgtttccacggctcgaacttgtgacttataggtcacacggaggcaacttcaccttaactagttagtaggtactataaatttcatgctccaaacataggtgcgtgcaagtagggaaaatcaaataggaattatgtctgtttgcctgattcttatcgatttgacggcgcactatgtgtaccaggattttttgggagagcatttcattgaaacttgaaaatttccttatctttttcttcagataaatgttccgttaccaagctcttgctctttctgtttgcctgaacagccctttctttttgccttagttcttacaattacttttataatcacttccttttcgttgtttgggctgcaggacaggaaaatgcttctgatactggatatgagtttatcaatcgacccatagctatgcgtcctctctttgacagcttgtatctgcagcaatatgttcttctctgctcccaggtgcttaaacttttagagagtatattctgaagttcgctagttgctctactatgtttcttggcattggtgtatgatttatgcatatgcacagcaggcctagctggaaaaataagagttaaagaaaataattcaacaaaactaaatcttagaaaaagctacctttttttctcataacatcacaggtttgcatcttaaaggtgctctctttggagaattagttgaaataaatttatgttatgcttctttctagtttcttctgaagatgaaatttgtgtctggttttgggggcatcagttaactacatcttttttgtgatcctcttgattagtgtaggatccagaagatctattttacattcatctcacagttagaacttttgcttttaatgtatacataagcaactatctatttaaccttcaaaaggagagggaaattagtaggtttatttatttttatatcagaaatcttttgaagatgttgcatttgtagcttttacaagtgtacgtgatgctctctgtttgatactttggcagtggaattccctctttctattgctcttttgcttttatatccttttcaagattctgatatcatattctggatgtagatagagggaggtttcagagacaagcctggcaagggtagagaccactaccatacgtgttactgtctaagtggtctttcaattgctcagtatagctggactaacgaagcagatgctccaccattacccagggatgtatttggtccttattctaccaacctgttagaacaaattcacccgctcttcaacgtagtgtt

SEQ ID NO: 35 - DNA, 1261 bp, Artificial Sequence
  PhGene2 reverse complement
cctattaaagataacaaggaagaagatgatatagatgaagatgcttttgaggcactgtttcagctactggaagaagatctgaaaaatgatgagctagctttggatgacgatgatataagtgaggaggacctcgcgaagcttgaacgtgaattggaagaggcactgaaggatgatgaactattaggagcaataggcaacattgctggtgaggaaattgagaatgacactgatgacgaagaaggggtagtagaagatgataacgaagaagaagaagaagaagaattacccgtgaatttgaaaaattggcaacttaaaaaactgggatatgccttgagaaagggccgacgtaagacaaatgtgagttgtgatctctatgttagtcttctcaaattaagatgttgcaatcgcactaccagatactcattattcttgaatcttaaagccaagttgcacccttgctctttgtatattccggcatcaagcacgaagcaaagaacatcaatccatgtgtctaaaattttaaattgcagtccgtatgtagcttcattcttcagattcaattgttgctgtagcattcaactggacatcttttttttggttaaaaaactggacatctactttatgcttgcaccattttcttggcattcattagctaaaactatatatcaatttgaacatgaagcatatcattaataattttgcatttggtgttcagctttcttctagataaagtcatcataagtatagtcggaagtttcagaacctgtgggcctgtggctgtaacataattggaacaacagaaaatatggaaatgccttattcttctgacctttgcttcctatttaaagagaatatttctaccactgctaattctgtatctgtaaattctatgtttccttccagataaaaaatcttgctgctgacctatgccttgatagagctgttgttttggaattacttcgtaatcctccaatgaaccttctgatgctgagcgaagcattaccagatgaacctgttcccagaatgatagagcctgaaagcaggctcctggagacttttcccatagagataagtgatgttccgaagactgaggctaaggtggaaacaccagttcatgtgatgcaaagtagttggtctgctcaaaagaggataaagaaagtccaattggaaacacttgaacaagtttatagaagatcgaagcggcctactgtgagtaccattacttctgtaattgtgtcaatggcccttaattccatgttag